(19) United States
(12) Patent Application Publication
(10) Pub. No.: US 2024/0376184 A1
(43) Pub. Date: Nov. 14, 2024

(54) IgA MONOCLONAL ANTIBODIES FOR TREATING FLAVIVIRUS INFECTION

(71) Applicants: The Research Foundation for the State University of New York, Albany, NY (US); THE GOVERNMENT OF THE UNITED STATES, AS REPRESENTED BY THE SECRETARY OF THE ARMY, FORT DETRICK, MD (US)

(72) Inventors: Adam WAICKMAN, Jamesville, NY (US); Adam WEGMAN, Syracuse, NY (US); Jeffrey CURRIER, Germantown, MD (US); Heather FRIBERG, Fort Detrick, MD (US); Michael MCCRACKEN, North Bethesda, MD (US); Gregory GROMOWSKI, Sandy Spring, MD (US)

(21) Appl. No.: 18/685,041

(22) PCT Filed: Aug. 22, 2022
(86) PCT No.: PCT/US2022/075260

§ 371 Date: Feb. 20, 2024

Related U.S. Application Data
(60) Provisional application No. 63/235,325, filed on Aug. 20, 2021.

(51) Int. Cl. C07K 16/10; A61P 31/04

(52) U.S. Cl. CPC C07K 16/1081; A61P 31/04; C07K 2317/24; C07K 2317/52; C07K 2317/76; C07K 2317/92

(57) ABSTRACT

The present disclosure relates to IgA antibodies and antigen binding fragments thereof and to cocktails of antibodies and antigen binding fragments that neutralize virus infection without contributing to antibody-dependent enhancement of dengue virus infection. The present disclosure also relates to immortalized B cells that produce, and to epitopes that bind to, such antibodies and antigen binding fragments.

### CROSS REFERENCE TO RELATED APPLICATIONS

This application claims the benefit of the filing date of U.S. Provisional Application No. 63/235,325 filed Aug. 20, 2021. The content of this earlier filed application is hereby incorporated by reference herein in its entirety.

### REFERENCE TO A SEQUENCE LISTING

This application contains a Sequence Listing in computer readable form, which is incorporated herein by reference.

### FIELD OF THE INVENTION

The present disclosure relates to immunoglobulin class switching or class-switch recombination, immunoglobulins that neutralize flavivirus infection and/or mitigate antibody-dependent enhancement (ADE) of flavivirus virus infection such as ADE associated with secondary heterologous dengue infections.

### BACKGROUND

Dengue virus (DENV) is one of the most widespread vector-borne viral pathogens in the world. Including four immunologically and genetically distinct serotypes (DENV −1, −2, −3, and −4), DENV is borne primarily by the tropical and subtropical mosquitoes Aedes aegypti and A. albopictus [1, 2]. DENV and its mosquito vectors can currently be found across Central and South America, South and South-East Asia, the Western Pacific, and sub-Saharan Africa, meaning 40% of the world's population is currently at risk of exposure and infection [1-3]. Consequently, an estimated 400 million DENV infections are thought to occur every year, resulting in 100 million clinically apparent infection [2]. Approximately 500,000 cases per year progress to severe dengue-characterized by thrombocytopenia, vascular leakage and hemorrhage-resulting in nearly 20,000 deaths [4-7].

A distinct epidemiological feature of dengue as compared to other flaviviral diseases is the increased risk for severe disease upon heterologous secondary infection [8]. While the risk factors associated with developing severe dengue upon secondary DENV exposure are complex and incompletely understood, the leading mechanistic explanation for this phenomenon is a process known as antibody-dependent enhancement (ADE) [9, 10]. ADE is thought to occur when poorly-neutralizing or sub-neutralizing concentrations of DENV-reactive IgG opsonizes DENV and facilitates its entry into permissive F_{c}γR-bearing cells [11]. Various lines of evidence support the association of ADE with severe dengue, including increased incidence of severe dengue in infants born to dengue-immune mothers [12-14]; increased viremia in interferon (IFN) receptor-deficient mice or non-primates passively immunized with anti-DENV antibodies [15, 16]; and increased incidence of severe dengue during the second of sequential/heterologous DENV outbreaks and in patients with a narrow range of preexisting anti-DENV antibody titers [17, 18]. Furthermore, in-vitro assessments of serum ADE activity in dengue-primed non-human primates have been shown to correlate with viral titers following heterologous attenuated DENV infection [19].

The increased risk of severe dengue upon secondary heterologous infection also presents a challenge to vaccine development as incomplete or waning vaccine-elicited immunity may place recipients at an increased risk of developing severe dengue should they be exposed following vaccination [20]. This is most significantly highlighted by the revelation that the only currently available DENV vaccine (DENGVAXIA®) fails to protect previously DENV naïve individuals from infection and can increase the risk of hospitalization with virologically confirmed dengue [21-23]. Accordingly, understanding the subtleties of both natural and vaccine-elicited DENV humoral immunity is critical for further the understanding of disease risk and infection-associated immunopathogenesis.

To date, the literature on dengue serology has overwhelmingly focused on the contribution of immunoglobulin isotypes IgM and IgG to functional dengue immunity and infection-associated immunopathogenesis. During both primary and secondary dengue infection, these isotype antibodies follow a highly predictable pattern of induction, with an IgM response preceding the rise of DENV-reactive IgG, and DENV-reactive IgG reaching significantly higher titers during secondary infection [24]. These characteristics, as well as the assumed importance of IgG-mediated ADE, have left the role of other serum antibody isotypes relatively unexamined. Notably, this includes IgA1, the second most prevalent antibody isotype in serum and one that has been suggested to play a unique and non-redundant role in many viral infections. Most work on DENV-reactive serum IgA has focused on its potential as a diagnostic tool [25], with a small literature examining DENV-reactive serum IgA as a possible correlate of severe disease [26-29].

The role for IgA1 during primary dengue was recently described, where IgA1 appears to be the dominant isotype-switched antibody expressed by DENV-elicited plasmablasts [30, 31]. While IgA expressing plasmablasts were also observed following secondary dengue, they constituted a significantly smaller fraction of the total infection-elicited immune response [30, 31]. Importantly, the IgA1 antibodies expressed by these DENV-elicited plasmablasts exhibited both DENV-binding and DENV-neutralization activity, comparable to what was observed for IgG isotype antibodies derived from contemporaneous samples [30].

Prior-art-of-interest includes U.S. Pat. No. 9,073,981 entitled Dengue virus neutralizing antibodies and use thereof (herein incorporated by reference). However, the reference is deficient in that it fails to identify the immunoglobulins of the present disclosure and uses thereof.

Accordingly, there is a continuing need for materials and methods for preventing flavivirus infection such as dengue virus without increasing the risk of antibody-dependent enhancement of infection.

### SUMMARY

The present disclosure is based, in part, on the observation of milder symptoms and lower viral burden typically associated with primary dengue relative to secondary dengue, and that DENV-reactive IgA1 plays a role in limiting DENV propagation and potentially the immune-mediated enhancement of disease. Below, isotype-switched antibodies show conversion of IgG1 to IgA1 does not impact the ability of a monoclonal antibody to either bind whole DENV virions or to neutralize DC-SIGN-dependent DENV infection of a susceptible cell line. Moreover, the inventors have found that IgG1 antibodies subjected to immunoglobulin class switching, wherein an Fc region include an IgA Fc domain or segment, provide DENV-reactive antibodies suitable for prophylaxis for flavivirus infection such as dengue virus and are capable of treating flavivirus disease such as dengue disease, and/or mediating ADE associated therewith.

In embodiments, immunoglobulins of the present disclosure include antibodies including the heavy chain or a segment of the heavy chain including an Fc region characterized as IgA Fc domain or segment, and cocktails of immunoglobulins including antibodies including the heavy chain or a segment of the heavy chain including an Fc region characterized as IgA Fc domain or segment, which neutralize dengue virus infection without contributing to antibody-dependent enhancement of dengue virus infection. Accordingly, in one aspect of the invention, the present disclosure includes a human antibody, an antibody variant, or an antigen binding fragment thereof, that neutralize a flavivirus virus infection such as dengue virus, wherein the antibody, antibody variant, or antigen binding fragment does not contribute to antibody-dependent enhancement of dengue virus infection.

In embodiments, the present disclosure includes treatments wherein adding antibodies of the present disclosure such as DENV-reactive monoclonal IgA1 to either an enhancing concentration of monoclonal IgG1 or to an enhancing dilution of dengue-immune plasma, antagonizes ADE in a dose-dependent fashion.

In embodiments, the present disclosure relates to an isolated monoclonal antibody, including: a heavy chain having an amino acid sequence of SEQ. ID NO. 1, wherein the heavy chain or a segment of the heavy chain includes an Fc region characterized as IgA Fc domain or segment. In embodiments, the IgA Fc domain is an IgA1 Fc domain.

In embodiments, the present disclosure relates to an isolated monoclonal antibody for targeting a fusion loop epitope of dengue virus, including: a heavy chain having an amino acid sequence having at least 90% sequence identity to SEQ. ID NO. 1, wherein the heavy chain or a segment of the heavy chain includes an Fc region characterized as IgA Fc domain; and a light chain having an amino acid sequence having at least 90% sequence identity to SEQ ID. NO. 2.

In some embodiments, the present disclosure relates to an isolated monoclonal antibody, including: a heavy chain including or consisting of an amino acid sequence of SEQ. ID NO. 1; and a light chain including or consisting of an amino acid sequence of SEQ ID NO: 2.

In some embodiments, the present disclosure relates to an isolated monoclonal antibody, including: a heavy chain having an amino acid sequence having at least 90% sequence identity to SEQ. ID NO. 1, wherein the heavy chain or a segment of the heavy chain includes an Fc region characterized as IgA Fc domain.

In some embodiments the present disclosure relates to a nucleic acid polymer encoding a monoclonal antibody, wherein the polymer includes or consists of SEQ. ID NO. 1 and/or a second nucleic acid polymer encoding a monoclonal antibody, wherein the second polymer includes or consists of SEQ. ID NO. 2.

In some embodiments, the present disclosure relates to a complementary deoxynucleotide (cDNA) sequence encoding an amino acid sequence having at least 90%, at least 95%, or at least 99% sequence identity to SEQ ID NO: 1.

In some embodiments, the present disclosure relates to a complementary deoxynucleotide (cDNA) sequence including or consisting of a nucleic acid sequence 10) of SEQ ID NO: 3.

In some embodiments, the present disclosure relates to a complementary deoxynucleotide (cDNA) sequence encoding an amino acid sequence having at least at least 90%, at least 95%, or at least 99% sequence identity to SEQ ID NO: 2.

In some embodiments, the present disclosure relates to a complementary deoxynucleotide (cDNA) sequence including or consisting of a nucleic acid sequence of SEQ ID NO: 4.

In some embodiments, the present disclosure relates to a method for preventing or treating a flavivirus virus infection such as dengue viral infection, the method including administering a therapeutically effective amount of the monoclonal antibody of the present disclosure to a subject in need thereof under conditions effective to treat the viral infection.

In some embodiments, the present disclosure relates to a method for preventing or treating antibody-dependent enhancement of a viral infection, the method including: administering a therapeutically effective amount of a monoclonal antibody including a heavy chain or a segment of the heavy chain including an Fc region characterized as IgA Fc domain to a subject in need thereof under conditions effective to treat the viral infection, wherein the IgA Fc domain is characterized as an isotypic commutation.

In some embodiments, the present disclosure relates to a method for preventing or treating antibody-dependent enhancement of a viral infection, the method including: administering a therapeutically effective amount of a monoclonal antibody including a heavy chain or a segment of the heavy chain including an Fc region characterized as IgA Fc domain to a subject in need thereof under conditions effective to treat the viral infection, wherein the IgA Fc domain is formed by class-switch recombination.

In some embodiments, the present disclosure relates to a method for preventing or treating antibody-dependent enhancement of a flavivirus infection, the method including: administering a therapeutically effective amount of an immunoglobulin such as: a) a monoclonal antibody encoded by a DNA polymer of the present disclosure; b) a an antibody encoded by one or more cDNAs of the present disclosure; or c) a monoclonal antibody of the present disclosure. In embodiments, the monoclonal antibody is disposed with a serum including one or more additional antibodies or fragments thereof directed against dengue virus or bind one or more epitopes of dengue virus.

In embodiments, the present disclosure includes a method for preventing or treating antibody-dependent enhancement of a viral infection, the method including administering a therapeutically effective amount of an antibody including a heavy chain or a segment of the heavy chain comprising an Fc region characterized as IgA Fc domain to a subject in need thereof under conditions effective to prevent or treat antibody-dependent enhancement of a viral infection.

In embodiments, the present disclosure includes a method for preventing or treating antibody-dependent enhancement of a viral infection, the method including administering a therapeutically effective amount of a monoclonal antibody to a subject 20) in need thereof under conditions effective to prevent or treat antibody-dependent enhancement of a viral infection, wherein the monoclonal antibody is characterized as an IgA isoform.

### BRIEF DESCRIPTION OF THE DRAWINGS AND SEQUENCE LISTING

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A, 1B and 1C depict an isotype conversion scheme of the present disclosure, DENV binding, and DENV neutralization capacity of VDB33 and VDB50 mAbs.

FIGS. 2A, 2B, 2C and 2D depict data indicating IgG1, but not IgA1, mediates ADE of DENV infection.

FIGS. 3A, 3B, 3C and 3D depict data and the fractional addition of DENV-reactive IgA1 significantly reduced the ADE activity observed in cultures containing either VDB33-IgG1 or VDB50-IgG1.

FIGS. 4A-4C depict Monoclonal IgA1 antagonizes ADE mediated by polyclonal DENV-immune plasma.

FIG. 5 depicts a gating scheme and representative plots from FlowNT assays.

FIG. 6 shows a gating scheme and representative plots from ADE assays.

FIGS. 7A, 7B and 7C show DENV −3 IgM, IgG, and IgA titers in DENV-immune plasma samples.

FIGS. 8A and 8B depict DENV −3 neutralization activity of DENV-immune plasma as assessed by FlowNT, and DENV −3 ADE activity of DENV-immune plasma as assessed by K562 infection.

FIG. 9 depicts Isotype distribution of plasmablasts captured during acute primary or secondary DENV infection by single cell RNA sequencing.

FIG. 10 shows isotype distribution of plasmablasts captured during acute secondary DENV infection in individuals that progressed to develop mild and severe dengue.

FIG. 11 depicts a proposed model for IgA antagonism of IgG-mediated DENV ADE.

FIG. 12 shows an analysis on samples collected around the day of fever abatement, prior to the critical phase of dengue where there is an increased probability of developing clinical manifestations of severe dengue.

FIGS. 13A, 13B, 13C, and 13D Example of IgG mediated enhancement of DENV infection in K562 cells expressing FcgR. Fold enhancement of infection calculated relative to infection level achieved in the absence of recombinant antibody

FIG. 14 depicts an annotated sequence of a synthesized antibody in accordance with the present disclosure.

FIG. 15 depicts an annotated sequence in accordance with the present disclosure.

FIG. 16 depicts DENV1_E_protein (Wp74, UniProt P17763.2) and target epitopes suitable for binding in accordance with the present disclosure.

FIG. 17 depicts VDB50_IgA_heavy_chain_AA and VDB50_IgA_light_chain_AA suitable for combination into an antibody suitable for use in accordance with the present disclosure.

FIG. 18 depicts DENV1_E_protein (Wp74, UniProt P17763.2) and target epitopes suitable for binding in accordance with the present disclosure.

SEQ ID NO: 1 depicts a peptide sequence for VDB33_IgA1_HC.
SEQ ID NO:2 depicts a peptide sequence for VDB33_IgA1_LC.
SEQ ID NO:3 depicts a nucleotide sequence for VDB33-IgA1_heavy_chain.
SEQ ID NO:4 depicts a nucleotide sequence for VDB33-IgA1_light_chain.
SEQ ID NO:5 depicts DENV1_E_protein including VDB33 target epitopes: W101, G106, L107, F108.
SEQ ID NO: 6 depicts a peptide sequence for VDB50-IgG1_heavy_chain.
SEQ ID NO:7 depicts a peptide sequence for VDB50-IgG1_light_chain.
SEQ ID NO:8 depicts a peptide sequence for VDB50_IgA_heavy_chain_nt.
SEQ ID NO:9 depicts a peptide sequence for VDB50_IgA_light_chain_nt.
SEQ ID NO: 10 depicts a peptide sequence for VDB50_IgA_heavy_chain_AA.
SEQ ID NO: 11 depicts a peptide sequence for VDB50_IgA_light_chain_AA.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The present disclosure relates to one or more immunoglobulins or functional fragments thereof, such as an antibody or functional fragment thereof having a heavy chain or a segment of the heavy chain including an Fc region characterized as IgA Fc domain or an IgA F_{c} segment. In embodiments, one or more non-IgA antibodies for targeting a flavivirus virus such as dengue virus are subjected to isotype-switching to include a heavy chain or a segment of the heavy chain including an Fc region characterized as IgA Fc domain. In embodiments, one or more immunoglobulins that neutralize flavivirus infection such as dengue and/or mitigate antibody-dependent enhancement (ADE) of flavivirus virus infection such as ADE associated with secondary heterologous dengue infections formed by class switching or class-switch recombination are provided. In embodiments, wild-type IgA antibodies are suitable for use in accordance with the present disclosure.

Advantages of the immunoglobulins, antibodies, or functional fragments thereof include excellent prophylaxis and treatment of flavivirus disease such as dengue disease associated with one or more dengue virus strains.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within +10% of the indicated value, whichever is greater.

The term “antibody” as used herein refers to an immunoglobulin molecule capable of specific binding to a target antigen or biomarker, such as a carbohydrate, polynucleotide, lipid, polypeptide, peptide etc., via at least one antigen recognition site (also referred to as a binding site), located in the variable region of the immunoglobulin molecule. In embodiments, the term “antibody” refers to a selective binding compound. In embodiments, antibodies, or functional fragments thereof may selectively bind or target any portion of one or more E proteins or fragments thereof of dengue virus, or a variant thereof. In embodiments, antibodies or functional fragments thereof refers to a compound that selectively binds to a fusion loop (FL) domain in a dengue viral envelope (E) protein associated with fusion of the viral membrane to a cellular membrane.

As used herein, the terms “bind” and “binding” generally refer to the non-covalent interaction between a pair of partner molecules or portions thereof (e.g., antigenic protein-binding partner complexes) that exhibit mutual affinity or binding capacity. In embodiments, binding can occur such that the partners are able to interact with each other to a substantially higher degree than with other, similar substances. This specificity can result in stable complexes (e.g., antigenic protein-binding partner complexes or bound biomarkers-of-interest) that remain bound during handling steps such as chromatography, centrifugation, filtration, and other techniques typically used for separations and other processes. In embodiments, the interaction between a target region of an antigenic protein and a binding partner that binds specifically thereto is a non-covalent interaction. In some instances, the interaction between a binding partner and a non-target region of an antigenic protein is a non-covalent interaction. However, in other instances, the interaction between a binding partner and a non-target region of an antigenic protein may be a covalent interaction. In embodiments, a protein complex comprising the antigenic protein and the binding partner may be contacted with a chemical crosslinking reagent that causes covalent bonds between the antigenic protein and the binding partner to be formed. In another example, the antigenic protein may contain a first reactive chemical moiety (handle) and the one or more binding partners may each contain a second reactive chemical moiety (handle), wherein the first and second chemical reactive moieties can react with each other to form a covalent bond. Exemplary reactive chemical moieties include those useable in “click” chemistry, which is a class of biocompatible small molecule reactions commonly used in bioconjugation, allowing the joining of substrates of choice with specific biomolecules. Click chemistry is not a single specific reaction, but refers to a way of generating products that follow examples in nature, which also generates substances by joining small modular units. In one example, the antigenic protein may have a first reactive chemical moiety such as a clickable handle like an azide, and the binding partner(s) could have a complementary reactive handle such as, for example a strained cyclooctyne, or vice versa. When these reactive chemical moieties come into proximity when the antigenic protein and the one or binding partners interact to form a protein complex, they can react with each other to form a covalently bond between the proteins.

As used herein the term “cDNA” refers to a DNA molecule that can be prepared by reverse transcription from an RNA molecule obtained from a eukaryotic or prokaryotic cell, a virus, or from a sample solution. In embodiments. cDNA lacks introns or intron sequences that may be present in corresponding genomic DNA In embodiments, cDNA may refer to a nucleotide sequence that corresponds to the nucleotide sequence of an RNA from which it is derived. In embodiments, cDNA refers to a double-stranded DNA that is complementary to and derived from mRNA.

As used herein the term “competitive inhibitor” refers to one or more substances that binds to or blocks another substance from participating in a reaction. Non-limiting examples of competitive inhibitors of the present disclosure include one or more antibodies of the present disclosure or fragments thereof that bind an envelope protein epitope or Dengue virus viral envelope glycoprotein, E, or one or more fusion loops disposed therein. In embodiments, antibodies of the present disclosure target the E dimer epitope (EDE), readily exposed at an E dimer interface over a region of a conserved fusion loop. In embodiments, antibodies of the present disclosure target a conserved fusion loop (FL) domain among dengue virus strains in a viral envelope (E) protein associated with fusion of the viral membrane to a cellular membrane.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

As used herein the terms “drug,” “drug substance,” “active pharmaceutical ingredient,” and the like, refer to a compound (e.g., immunoglobulin or antibody) that may be used for treating a subject in need of treatment.

As used herein the term “excipient” or “adjuvant” refers to any inert substance.

As used herein the terms “drug product,” “pharmaceutical dosage form,” “dosage form,” “final dosage form” and the like, refer to a pharmaceutical composition that is administered to a subject in need of treatment and generally may be in the form of inhalers, tablets, capsules, sachets containing powder or granules, liquid solutions or suspensions, patches, and the like.

As used herein “dengue virus” refers to one of any of four related viruses: Dengue virus 1, 2, 3, and 4. In embodiments, dengue virus includes a single strand of RNA, or positive-sense RNA that can be directly translated into proteins. In embodiments, the viral genome encodes ten genes. In embodiments, the genome is translated as a single, long polypeptide and then cut into ten proteins. In embodiments, the dengue virus genome encodes three structural (capsid [C], membrane [M], and envelope [E]) and seven nonstructural (NS1, NS2A, NS2B, NS3, NS4A, NS4B, and NS5) proteins. In embodiments, the dengue virus includes a highly conserved fusion loop (FL) domain in the viral envelope (E) protein associated with fusion of the viral membrane to a cellular membrane.

As used herein the term “flavivirus” refers to any of a group of RNA viruses, mostly having arthropod vectors, that cause a number of serious human diseases including yellow fever, dengue, various types of encephalitis, and hepatitis C.

As used herein the term “fragment” means a polypeptide having one or more (e.g., several) amino acids absent from the amino and/or carboxyl terminus of a mature polypeptide or domain. In embodiments, a fragment is able to bind to Dengue virus viral envelope glycoprotein, E, or one or more fusion loops disposed therein. In embodiments, a fragment is able to target fusion loop (FL) domain in the viral envelope (E) protein associated with fusion of the viral membrane to a cellular membrane. In embodiments, a fragment contains at least 70% to 99%, at least 90% to 99% or about 95 to 99% of the number of amino acids of the mature polypeptide of SEQ ID NO: 1 and/or SEQ ID NO:2.

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences.

Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). In embodiments, a polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The term “isolated” means a substance in a form or environment that does not occur in nature. Non-limiting examples of isolated substances include (1) any non-naturally occurring substance, (2) any substance such as a variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

The term “mature polypeptide” means a polypeptide in its final form following translation and any post-translational modifications, such as N-terminal processing. C-terminal truncation, glycosylation, etc.

The term “nucleotide” refers to a ribonucleotide or a deoxyribonucleotide or modified form thereof, as well as an analog thereof.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded polynucleotides.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may include additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

In embodiments, when calculating sequence identity between a DNA sequence and an RNA sequence, T residues of the DNA sequence align with, and can be considered “identical” with. U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm, and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

The term “substantially purified,” as used herein, refers to a component of interest that may be substantially or essentially free of other components which normally accompany or interact with the component of interest prior to purification. In embodiments, a component of interest may be “substantially purified” when the preparation of the component of interest contains less than about 30%, less than about 25%, less than about 20%, less than about 15%, less than about 10%, less than about 10) 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. In embodiments, a component of interest includes a virus-of-interest, such as dengue virus or a variant thereof.

“Substantially similar” refers to nucleic acid molecules wherein changes in one or more nucleotide bases result in substitution of one or more amino acids, but do not affect the functional properties of the protein encoded by the DNA sequence. “Substantially similar” also refers to nucleic acid molecules wherein changes in one or more nucleotide bases do not affect the ability of the nucleic acid molecule to mediate alteration of gene expression by antisense or co-suppression technology.

“Substantially similar” also refers to modifications of the nucleic acid molecules of the instant disclosure (such as deletion or insertion of one or more nucleotide bases) that do not substantially affect the functional properties of the resulting transcript vis-a-vis the ability to mediate alteration of gene expression by antisense or co-suppression technology or alteration of the functional properties of the resulting protein molecule. The disclosure encompasses more than the specific exemplary sequences.

As used herein the term “pharmaceutically acceptable” substances refers to those substances, such as e.g., antibodies of the present disclosure and functional fragments thereof, which are within the scope of sound medical judgment suitable for use in contact with the tissues of subjects without undue toxicity, irritation, allergic response, and the like, and effective for their intended use.

As used herein the term “pharmaceutical composition” refers to the combination of one or more drug substances such as e.g., antibodies of the present disclosure or functional fragments thereof and one or more excipients and one or more pharmaceutically acceptable vehicles with which the one or more antibodies or functional fragments thereof is administered to a subject.

As used herein the term “pharmaceutically acceptable vehicle” refers to a diluent, adjuvant, excipient or carrier with which a compound, such as e.g., an antibody of the present disclosure or functional fragment thereof, is administered.

As used herein the term “prevent”, “preventing” and “prevention” of dengue means (1) reducing the risk of a patient who is not experiencing symptoms of dengue virus infection from developing dengue, or (2) reducing the frequency of, the severity of, or a complete elimination of dengue symptoms already being experienced by a subject.

The term “prophylactically effective amount,” as used herein, refers to that amount of a composition, such as e.g., antibody of the present disclosure or a functional fragment thereof, administered to a subject which will relieve to some extent one or more of the symptoms of a disease, likelihood of becoming diseased, or condition or disorder being treated. In such prophylactic applications, such amounts may depend on the subject's state of health, weight, and the like. It is considered well within the skill of the art for one to determine such prophylactically effective amounts by routine experimentation, including, but not limited to, a dose escalation clinical trial.

The term “recombinant” when used herein to characterize a DNA sequence such as a plasmid, vector, or construct refers to an artificial combination of two otherwise separated segments of sequence, e.g., by chemical synthesis and/or by manipulation of isolated segments of nucleic acids by genetic engineering techniques. As used herein the term “subject” includes humans, animals or mammals. The terms “subject” and “patient” may be used interchangeably herein.

As used herein, the term “selective binding compound” refers to a compound that selectively binds to any portion of one or more target proteins.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “target protein” refers to a molecule or a portion of a protein capable of being bound by a selective binding compound. In certain embodiments, a target protein is one or more E proteins or fragments thereof of dengue virus (including variants thereof). In embodiments, a target protein is a fusion loop (FL) domain in a dengue viral envelope (E) protein associated with fusion of the viral membrane to a cellular membrane.

As used herein the term “therapeutically effective amount” means the amount of a compound that, when administered to a subject for treating or preventing flavivirus infection such as a dengue virus infection, is sufficient to effect such treatment or prevention of flavivirus disease such as dengue and related symptoms. A “therapeutically effective amount” can vary depending, for example, on the compound, the severity of the dengue infection, the etiology of the dengue infection, one or more prior dengue infections, the age of the subject to be treated, comorbidities of the subject to be treated, existing health conditions of the subject, and/or the weight of the subject to be treated. A “therapeutically effective amount” is an amount sufficient to alter the subjects' natural state.

As used herein the term “treat”, “treating” and “treatment” of flavivirus disease such as dengue means an intervention for reducing the frequency of symptoms of flavivirus disease such as dengue, eliminating the symptoms of flavivirus disease such as dengue, avoiding or arresting the development of symptoms of flavivirus disease such as dengue, ameliorating or curing an existing or undesirable symptom caused by flavivirus disease such as dengue, and/or reducing the severity of symptoms of flavivirus disease such as dengue.

In embodiment the term “variant” means a polypeptide including an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more (e.g., several) amino acids, e.g., 1-10 amino acids, adjacent to the amino acid occupying a position.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997);

and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DETAILED DESCRIPTION OF CERTAIN EMBODIMENTS

In embodiments, the present disclosure includes one or more immunoglobulins or functional fragments thereof, such as an antibody or functional fragment thereof having a heavy chain or a segment of the heavy chain including an Fc region characterized as IgA Fc domain or an IgA F_{c} segment suitable for prophylaxis and treatment of flavivirus disease such as dengue disease associated with one or more dengue virus strains.

In embodiments, the present disclosure includes an antibody, including: a heavy chain having an amino acid sequence of SEQ. ID NO. 1, wherein the heavy chain or a segment of the heavy chain includes an Fc region characterized as IgA Fc domain. In embodiments, the IgA Fc domain is an IgA1 Fc domain, or IgA2 Fc domain. In embodiments, the antibody is characterized as isolated and/or monoclonal. In embodiments, the antibody is chimeric or humanized. In some embodiments, an isolated monoclonal antibody includes: a light chain having an amino acid sequence of SEQ ID. NO. 2. In embodiments, an antibody, such as an isolated monoclonal antibody binds an epitope of a Deng virus, wherein the epitope is characterized as a loop.

In embodiments, the Fc region characterized as IgA Fc domain includes

or consists of the amino acids of SEQ ID NO: 1. In embodiments, the Fc region characterized as IgA Fc domain includes or consists of the amino acids between S31 and Y475 of SEQ ID NO. 1, an amino acid segment between G51 and Y475 of SEQ ID NO: 1, an amino acid segment between L101, or an amino acid segment between A181 and Y475, wherein SEQ ID NO: 1 is used for numbering.

In embodiments, an antibody, such as an isolated monoclonal antibody or functional fragment thereof is suitable for targeting a fusion loop epitope of dengue virus. In embodiments, such antibodies include a heavy chain having an amino acid sequence having at least 90% sequence identity to SEQ. ID NO. 1, wherein the heavy chain or a segment of the heavy chain includes an Fc region characterized as IgA Fc domain; and a light chain having an amino acid sequence having at least 90% sequence identity to SEQ ID. NO. 2. In embodiments, the heavy chain includes an amino acid sequence having at least 95%, 97%, 98%, 99% sequence identity to SEQ. ID NO. 1. In embodiments, the light chain includes an amino acid sequence having at least 95%, 97%, 98%, 99% sequence identity to SEQ. ID NO. 2.

In embodiments, the immunoglobulins of the present disclosure include an isolated monoclonal antibody, including: a heavy chain consisting of an amino acid sequence of SEQ. ID NO. 1; and a light chain consisting of an amino acid sequence of SEQ ID NO: 2. In embodiments, the isolated monoclonal antibody binds a fusion loop epitope (FLE) of a Dengue virus.

30) In embodiments, the immunoglobulins of the present disclosure include an isolated monoclonal antibody, including: a heavy chain having an amino acid sequence having at least 90% sequence identity to SEQ. ID NO. 1, wherein the heavy chain or a segment of the heavy chain includes an Fc region characterized as IgA Fc domain. In embodiments, the isolated monoclonal antibody binds an epitope of a Dengue virus, wherein the epitope is characterized as a loop.

In embodiments, the present disclosure includes a nucleic acid or nucleic acid polymer encoding a monoclonal antibody, wherein the polymer includes or consists of SEQ. ID NO. 1. In embodiments, the present disclosure includes a nucleic acid polymer encoding a monoclonal antibody, wherein the polymer includes or consists of SEQ. ID NO. 2.

In embodiments, the present disclosure includes a complementary deoxynucleotide (cDNA) sequence encoding an amino acid sequence or antibody of the present disclosure. In embodiments, the present disclosure includes a complementary deoxynucleotide (cDNA) sequence encoding an amino acid sequence having at least at least 90%, at least 95%, or at least 99% sequence identity to SEQ ID NO: 1. In embodiments, the present disclosure includes a complementary deoxynucleotide (cDNA) sequence including or consisting of a nucleic acid sequence of SEQ ID NO: 3.

In embodiments, the present disclosure includes a complementary deoxynucleotide (cDNA) sequence encoding an amino acid sequence having at least at least 90%, at least 95%, or at least 99% sequence identity to SEQ ID NO: 2. In embodiments, the present disclosure includes a complementary deoxynucleotide (cDNA) sequence including or consisting of a nucleic acid sequence of SEQ ID NO: 4.

In some embodiments, the present disclosure includes a method for preventing or treating a dengue viral infection, the method including administering a therapeutically effective amount of the immunoglobulins described herein, including a monoclonal antibody, to a subject in need thereof under conditions effective to treat the viral infection. In embodiments, the method is used for preventing or treating antibody-dependent enhancement of a viral infection.

In some embodiments, the present disclosure includes a method for preventing or treating antibody-dependent enhancement of a viral infection, the method including: administering a therapeutically effective amount of a monoclonal antibody including a heavy chain or a segment of the heavy chain including an Fc region characterized as IgA Fc domain to a subject in need thereof under conditions effective to treat the viral infection, wherein the IgA Fc domain is characterized as an isotypic commutation. In embodiments, the viral infection is any one of respiratory syncytial virus (RSV), influenza, and coronavirus infection, or a combination thereof. In embodiments, the viral infection is a flavivirus infection. In embodiments, the flavivirus infection is a Zika virus, or a dengue virus.

In some embodiments, the present disclosure includes a method for preventing or treating antibody-dependent enhancement of a viral infection, the method including: administering a therapeutically effective amount of a monoclonal antibody including a heavy chain or a segment of the heavy chain including an Fc region characterized as IgA Fc domain to a subject in need thereof under conditions effective to treat the viral infection, wherein the IgA Fc domain is formed by class-switch recombination.

In some embodiments, the present disclosure includes a method for preventing or treating antibody-dependent enhancement of a flavivirus infection, the method including administering a therapeutically effective amount of: a) a monoclonal antibody encoded by a DNA polymer of the present disclosure; b) a monoclonal antibody encoded by a cDNA of the present disclosure; or c) a monoclonal antibody of the present disclosure. In embodiments, the monoclonal antibody is disposed with a serum including one or more additional antibodies or fragments thereof directed against dengue virus or bind one or more epitopes of dengue virus.

In some embodiments, the present disclosure to IgA1 class-switched monoclonal antibody therapy, which may be an effective therapy for preventing dengue virus infection without risking immune mediated enhancement of disease due to waning antibody titers. Further, the present invention relates to the fields of immunology and virology, including methods of assessing monoclonal antibody and plasma DENV-reactivity using DENV-capture ELISA protocol, assessing neutralizing titers of monoclonal antibodies and heat-inactivated plasma using a neutralization assay, and quantification of DENV −3 infection using ADE assay.

In some embodiments, e the present disclosure includes an Immunoglobulin A1 (IgA1) monoclonal antibody; an Immunoglobulin G1 (IgG1) monoclonal antibody; a method of administering a therapeutic treatment for DENV; a method of making a therapeutic treatment for DENV; isolated isotype-switched monoclonal antibodies comprising vdb33 IgA1 and vdb50 IgG1; a method of quantification of DENV −3 infection using in vitro ADE assay; and an IgA1 class-switched monoclonal antibody therapy, and combinations of these.

In some embodiments, the present disclosure includes a method for preventing or treating antibody-dependent enhancement of a viral infection, the method including: administering a therapeutically effective amount of an antibody including a heavy chain or a segment of the heavy chain comprising an Fc region characterized as IgA Fc domain to a subject in need thereof under conditions effective to prevent or treat antibody-dependent enhancement of a viral infection. In embodiments, the viral infection is any one of respiratory syncytial virus (RSV), influenza, and coronavirus infection, or a combination thereof. In embodiments, the viral infection is a flavivirus infection. In embodiments, the flavivirus infection is a Zika virus. In embodiments, the flavivirus infection is a dengue virus. In embodiments, the method further includes increasing the concentration of antibodies characterized as IgA in a serum, such as a pharmaceutically acceptable serum, including one or more additional antibodies. In embodiments, the antibody is a wild-type IgA isoform that binds dengue protein E. In embodiments the antibody is monoclonal and or characterized as pharmaceutically acceptable. In embodiments, the antibody is synthetic, formed by recombinant technology, or characterized as isolated and/or pharmaceutically acceptable. In embodiments, antibodies of the present disclosure are disposed within a pharmaceutically acceptable compositions and may include additional excipients. In embodiments, antibodies of the present disclosure are disposed within a pharmaceutically acceptable vehicle suitable for administration to a subject in need thereof.

In embodiments, the present disclosure includes a method for preventing or treating antibody-dependent enhancement of a viral infection, the method including administering a therapeutically effective amount of a monoclonal antibody to a subject in need thereof under conditions effective to prevent or treat antibody-dependent enhancement of a viral infection, wherein the monoclonal antibody is characterized as an IgA isoform. In embodiments, the monoclonal antibody includes a heavy chain or a segment of the heavy chain comprising an Fc region characterized as IgA Fc domain. In embodiments, the Fc region characterized as IgA Fc domain includes or consists of the amino acids of SEQ ID NO: 1. In embodiments, the Fc region characterized as IgA Fc domain includes or consists of the amino acids between S31 and Y475 of SEQ ID NO. 1, an amino acid segment between G51 and Y475 of SEQ ID NO: 1, an amino acid segment between L101, or an amino acid segment between A181 and Y475, wherein SEQ ID NO: 1 is used for numbering.

In embodiments, the present disclosure includes a method for preventing or treating antibody-dependent enhancement of a viral infection, the method including administering a therapeutically effective amount of a polypeptide to a subject in need thereof under conditions effective to prevent or treat antibody-dependent enhancement of a viral infection, wherein the polypeptide is characterized as an IgA antibody isoform. In embodiments, the polypeptide is an immunoglobulin 5 and includes a heavy chain or a segment of the heavy chain comprising an Fc region characterized as IgA Fc domain.

### Example I

### Materials and Methods

Viruses: DENV −3 (strain CH53489) propagated in Vero cells were utilized for ELISA, FlowNT50, and ADE assays. Virus for ELISA was purified by ultracentrifugation through a 30% sucrose solution and the virus pellet was resuspended in PBS.

Cell lines: Human K562 cells were maintained in IMDM supplemented with 10% FBS, penicillin, and streptomycin. U937-DC-SIGN cells were maintained in RPMI supplemented with 10% FBS, L-glutamine, penicillin, and streptomycin.

Monoclonal antibodies and serum: The variable regions from the heavy and light chains were codon optimized, synthesized in vitro and subcloned into a pcDNA3.4 vector containing the human IgG1 or IgA1 Fc region by a commercial partner (Genscript). Transfection grade plasmids were purified by maxiprep and transfected into a 293-6E expression system. Cells were grown in serum-free 20 FreeStyle 293 Expression Medium (Thermo Fisher), and the cell supernatants collected on day 6 for antibody purification. Following centrifugation and filtration, the cell culture supernatant was loaded onto an affinity purification column, washed, eluted, and buffer exchanged to the final formulation buffer (PBS). Antibody lot purity was assessed by SDS-PAGE, and the final concentration determined by 280 nm absorption. The clonotype information for all monoclonal antibodies generated as part of this study is listed in Table 1.

TABLE 1
Sequence information of DENV-reactive monoclonal antibodies

| | | |
|---|---|---|
| Clone name | VDB33 | VDB50 |
| Parental Isotype | IgG1 | IgA1 |
| Infecting Serotype | DENV-3 | DENV-1 |
| Primary/Secondary | Secondary | Primary |
| H_{c} CDR3aa | CARLLQYKWNWLFDPW | CAKASQMATVFIDYW |
| H_{c} V | IGHV4-39*01 | IGHV3-23*03 |
| H_{c} D | IGHD1-7*01 | IGHD5-24*01 |
| H_{c} J | IGHJ5*02 | IGHJ4*02 |
| H_{c} Total SHM | 26 | 13 |
| L_{c} CDR3aa | CQVWDSDSDHPVF | CQSYDSSLSGGVF |
| L_{c} V | IGLV3-21*03 | IGLV1-40*01 |
| L_{c} J | IGLJ3*02 | IGLJ3*02 |
| L_{c} Total SHM | 14 | 8 |
| Target residues | W101, G106, L107, F108 | G100, W101, F108 |
| E protein epitope | Fusion loop | Fusion loop |

Dengue IgG antibody positive plasma was purchased from SeraCare. Donor ID and batch numbers are shown in Supplemental Table 1.

SUPPLEMENTAL TABLE 1
Dengue immune plasma used in this study

| Supplier | Product # | Donor ID | Batch number |
|---|---|---|---|
| SeraCare | 0325-0014 | BD250524 | 10127363 |
| SeraCare | 0325-0014 | BD250525 | 10127364 |
| SeraCare | 0325-0014 | BD250535 | 10127374 |
| SeraCare | 0325-0014 | BD250543 | 10127383 |

DENV-capture ELISA: Monoclonal antibody and plasma DENV-reactivity was assessed using a 4G2 DENV capture ELISA protocol. In short, 96 well NUNC MaxSorb flat-bottom plates were coated with 2 μg/ml flavivirus group-reactive mouse monoclonal antibody 4G2 (Envigo Bioproducts, Inc.) diluted in borate saline buffer. Plates were washed and blocked with 0.25% BSA+1% Normal Goat Serum in PBS after overnight incubation. DENV −3 (strain CH53489) diluted in blocking buffer was captured for 2 hr, followed by extensive washing with PBS+0.1% Tween 20. Serially diluted monoclonal antibody samples were incubated for 1 hr at RT on the captured virus, and DENV-specific antibody binding quantified using anti-human IgG HRP (Sigma-Aldrich, SAB3701362). Secondary antibody binding was quantified using the TMB Microwell Peroxidase Substrate System (KPL, cat. #50-76-00) and Synergy HT plate reader (BioTek, Winooski, VT). Antibody data were analyzed by nonlinear regression (One site total binding) to determine EC50 titers in GraphPad Prism 8 (GraphPad Software, La Jolla, CA).

Neutralization Assay: Neutralizing titers of monoclonal antibodies and heat-inactivated plasma were assessed using a flow cytometry-based neutralization assay in U937 cells expressing DC-SIGN as previously described [32, 33]. Four-fold dilutions of antibody or sera were mixed with an equal volume of virus diluted to a concentration to achieve 10%-15% infection of U937-DC-SIGN cells in the absence of antibody. The antibody/virus mixture was incubated for 1 h at 37° C., after which an equal volume of medium (RPMI-1640 supplemented with 10% FBS, 1% penicillin/streptomycin, 1% I-glutamine (200 mM) containing 5×10⁴ U937-DC-SIGN cells was added to each well and incubated 18-20 hr overnight in a 37° C., 5% CO2, humidified incubator. Following overnight incubation, the cells were fixed with IC Fixation Buffer (Invitrogen, 00-82222-49), permeabilized using IC Permeabilization Buffer (Invitrogen, 00-8333-56) and immunostained with flavivirus group-reactive mouse monoclonal antibody 4G2 (Envigo Bioproducts, Inc.), and secondary polyclonal goat anti-mouse IgG PE-conjugated antibody (#550589, BD Biosciences). The percentage of infected cells were quantified on a BD Accuri C6 Plus flow cytometer (BD Biosciences). Data were analyzed by nonlinear regression to determine 50% neutralization titers in GraphPad Prism 8 (GraphPad Software, La Jolla, CA).

ADE Assay: In vitro antibody-dependent enhancement (ADE) of DENV-3 infection was quantified as previously described [30, 34]. Four-fold serial dilutions of antibody or heat-inactivated sera were incubated with virus (in sufficient amounts to infect 10%-15% of U937-DC-SIGN cells) at a 1:1 ratio for 1 h at 37° C. This mixture was then added to a 96-well plate containing 5×10⁴ K562 cells per well in duplicate. Cells were cultured for 18-20 hr overnight in a 37° C., 5% CO2, humidified incubator. Processing and quantification continued as outlined in the FlowNT50 methods.

Statistical Analysis: All statistical analysis was performed using GraphPad Prism 8 Software (GraphPad Software, La Jolla, CA). A P-value <0.05 was considered significant.

### Results

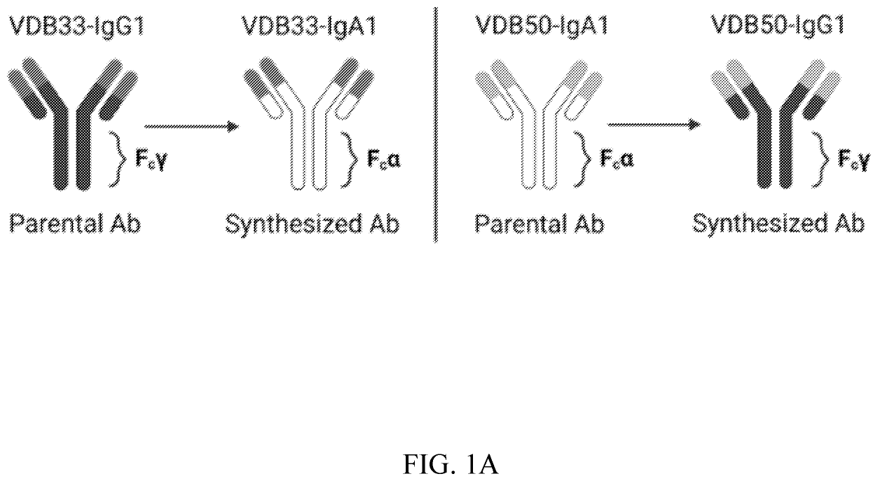

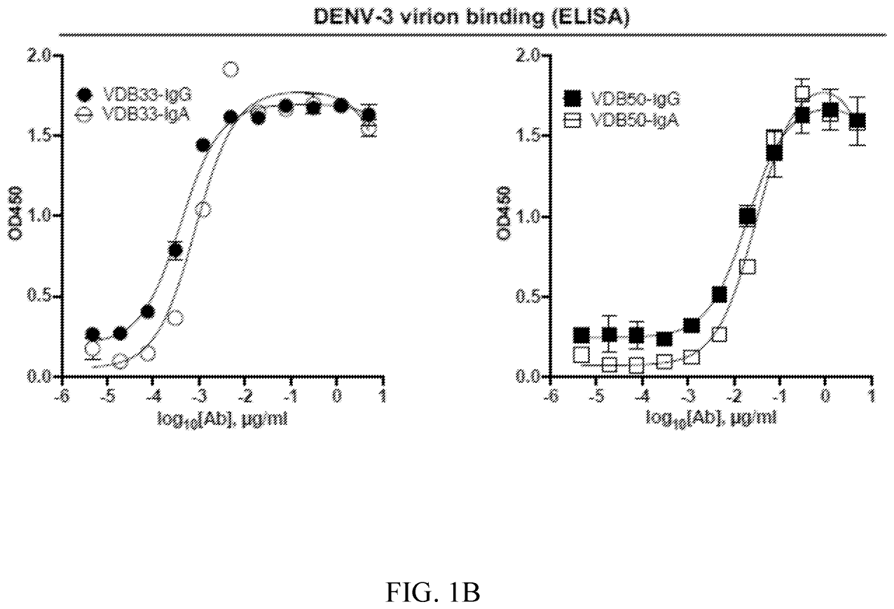

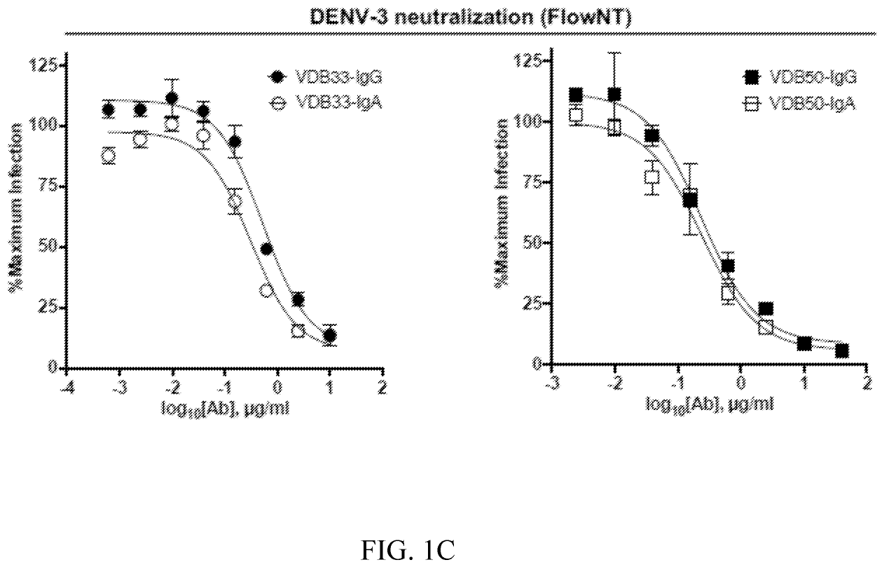

DENV binding and neutralizing is unaffected by antibody F_{c} isotype. To assess the potential contribution of DENV-reactive IgA1 to a functional anti-DENV humoral immune response, two pairs of DENV-reactive monoclonal antibodies with either an IgG1 or an IgA1 Fc domain (FIG. 1A). Both mAbs selected for this analysis were previously determined to bind the fusion loop of the DENV E protein and to react with all 4 DENV serotypes [30]. However, VDB33 was initially identified as an IgG1 clone, while VDB50 was discovered as an IgA1 clone (Table 1). This cross-conversion strategy was chosen so as to determine if the native F_{c} configuration of a given antibody influenced its functionality as either an IgG1 or IgA1 protein product.

The DENV-binding capacity of the IgG1 and IgA1 versions of VDB33 and VDB50 was initially assessed with a DENV virion-capture ELISA. For this analysis, DENV −3 was chosen as the prototypic DENV serotype as previous work demonstrated that the IgG1 versions of both VDB33 and VDB50 exhibited significant DENV −3 reactivity [30]. It was found that both VDB33 and VDB50 exhibited potent DENV −3 binding activity with VDB33 demonstrating ˜200 fold higher affinity for DENV −3 than VDB50 (FIG. 1A, Table 2).

TABLE 2
Functional characteristics isotype-switched monoclonal antibodies

| Clone name | VDB33 IgG | VDB33 IgA | VDB50 IgG | VDB50 IgA |
|---|---|---|---|---|
| Kd (ng/mL) | 0.3962 | 0.8296 | 19.92 | 31.12 |
| IC50 (ug/mL) | 0.5339 | 0.3139 | 0.2391 | 0.2439 |

However, the DENV-binding capacity of the two mAbs was not impacted by their conversion to either an IgG1 or IgA1 format (FIG. 1A, Table 2). Furthermore, this cross-conversion of VDB33 and VDB50 to either an IgG1 or IgA1 format minimally impacted the DENV −3 neutralization activity of the clones when assessed using a flow cytometry-based neutralization assay (FIG. 1B, Table 2). These results indicate that both IgG1 and IgA1 isotype antibodies are equally capable of binding and neutralizing DENV, reaffirming that antibody epitope/paratope interactions occur independently of an antibody's Fc domain.

DENV-reactive IgA1 is incapable of mediating ADE. Having demonstrated that the antigen binding and neutralization capacity of DENV-reactive monoclonal antibodies is negligibly impacted by the isotype of the construct, determination of whether the infection-enhancing capability of these antibodies was impacted by their isotype conversion was investigated. To this end, a K562-based ADE assay was used, wherein antibody/DENV immune complexes were pre-formed and added to the F_{c}-receptor expressing K652 cell line to assess the ability of defined antibody complexes to enhance DENV infection.

More specifically, FIG. 1A and FIG. 1B depict an isotype conversion scheme of the present disclosure, DENV binding, and DENV neutralization capacity of VDB33 and VDB50 mAbs. FIG. 1A depicts a schematic of isotype conversion of VDB33 and VDB50 from respective parental isotypes, indicating conservation of antigen-binding domains and alteration of Fc domains. FIG. 1B depicts DENV −3 binding capability of VDB33-IgG1, VDB33-IgA1, VDB50-IgG, and VDB50-IgA measured by DENV virus-capture ELISA. FIG. 1C depicts DENV −3 neutralization capability of VDB33-IgG, VDB33-IgA, VDB50-IgG, and VDB50-IgA as assessed by FlowNT. Neutralization data are presented as a percent of the positive (no neutralizing mAb) control for each replicate. Error bars +/− SEM.

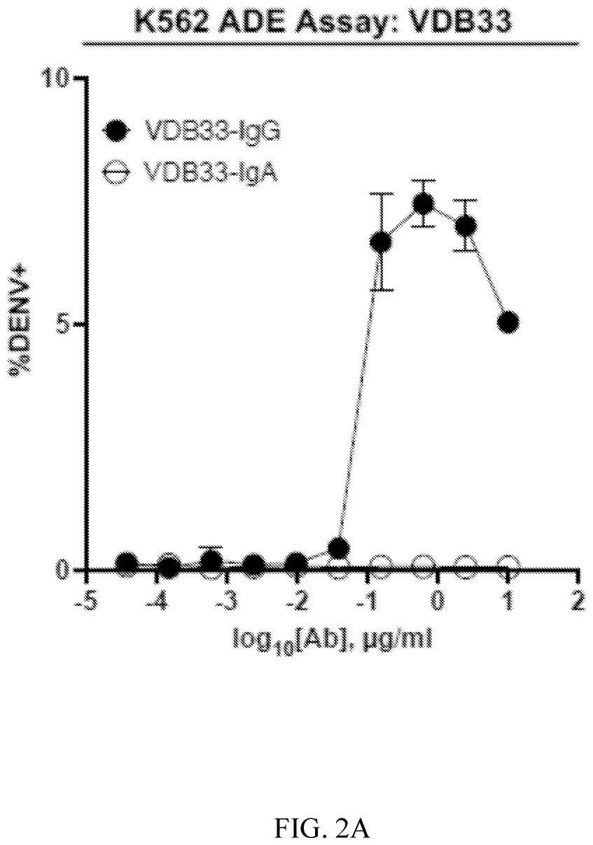

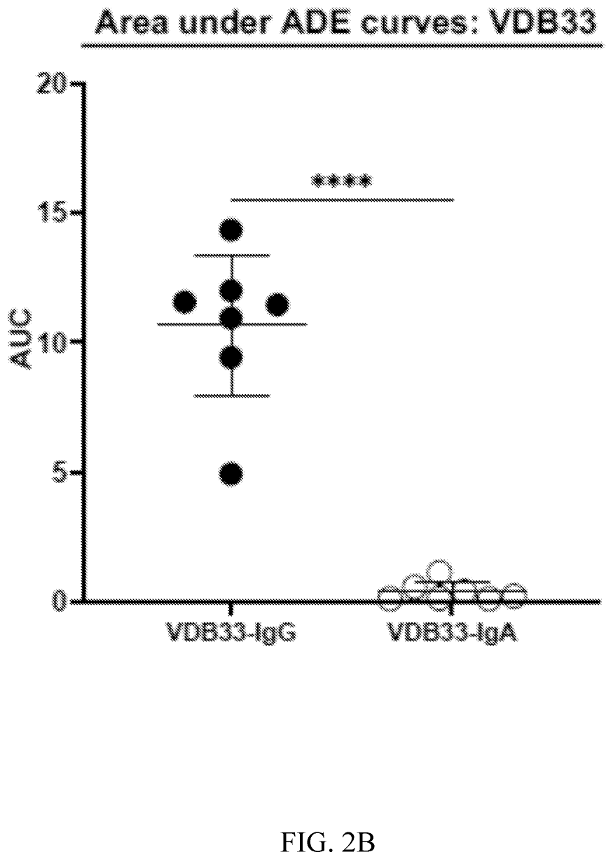

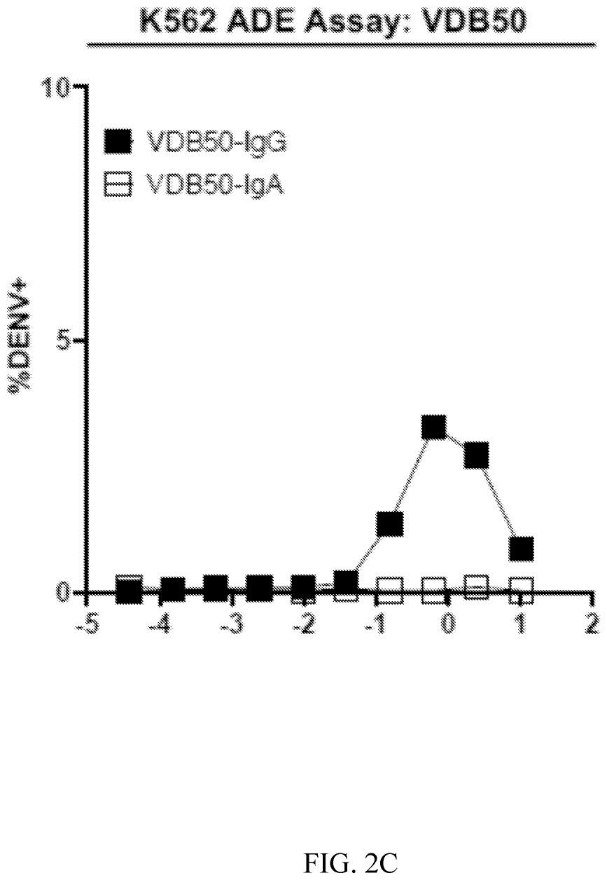

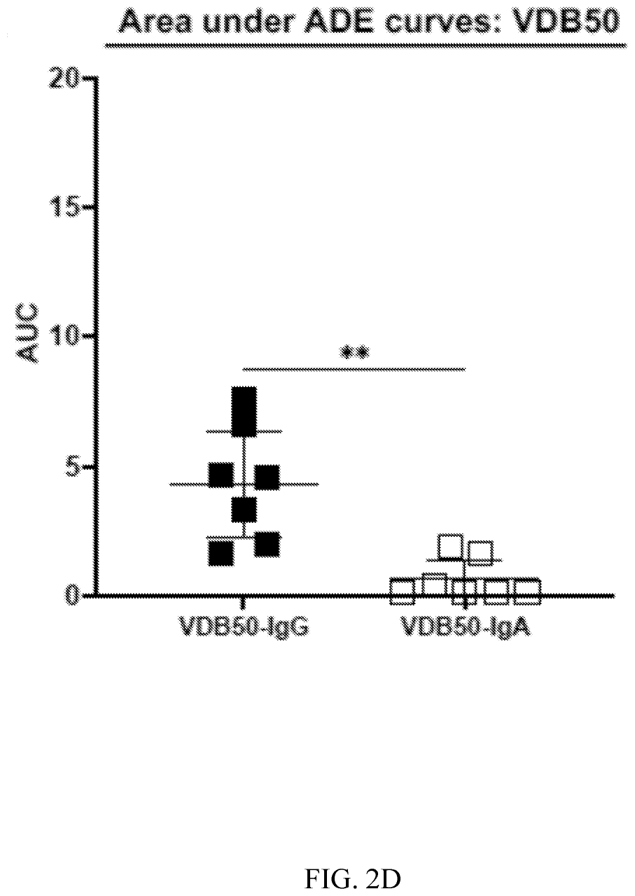

The IgG1 versions of both VDB33 and VDB50 exhibited potent infection-enhancing activity in the K562 ADE assay, with both antibodies capable of facilitating DENV infection/enhancement in a dose-dependent fashion (See FIG. 2A and FIG. 2B). Consistent with their relative EC₅₀/IC₅₀ values, VDB33-IgG1 exhibited notably higher ADE activity than VDB50-IgG1, but with the peak of ADE activity occurring at a similar antibody concentration. However, no infection enhancement was observed when the same assay was performed with either VDB33-IgA1 or VDB50-IgA1 (See FIG. 2A and FIG. 2B). This was despite the fact that these IgA1 isotype antibodies exhibit nearly identical virus binding and neutralization activity as their IgG1 counterparts, underlining the obligate role of an antibody's Fc domain in determining the ADE potential of an antibody.

More specifically, FIGS. 2A-2D depict data indicating IgG1, but not IgA1, mediates ADE of DENV infection. FIG. 2A depicts ADE activity of VDB33-IgG and VDB33-IgA against DENV −3 in K562 cells. FIG. 2B depicts AUC values of 7 independent replicates of DENV −3 ADE assay with VDB33-IgG and VDB33-IgA. FIG. 2C depicts ADE activity of VDB50-IgG and VDB50-IgA against DENV −3 in K562 cells. FIG. 2D depicts AUC values of 7 independent replicates of DENV −3 ADE assay with VDB50-IgG and VDB50-IgA. Error bars +/− SEM. ** p<0.01, **** p<0.0001, unpaired t test.

DENV-reactive IgA1 antagonizes IgG1 mediated enhancement of DENV infection. In light of the inability of VDB33-IgA1 and VDB50-IgA1 to facilitate ADE of DENV −3, how DENV-reactive IgG1 and IgA1 behave in a polyclonal/competitive setting was determined. IgG1 and IgA1 antibodies are never found in isolation in a dengue immune individual, so determining how these antibodies function in a complex/poly-immune setting is critical for understanding their potential contribution to function anti-DENV immunity.

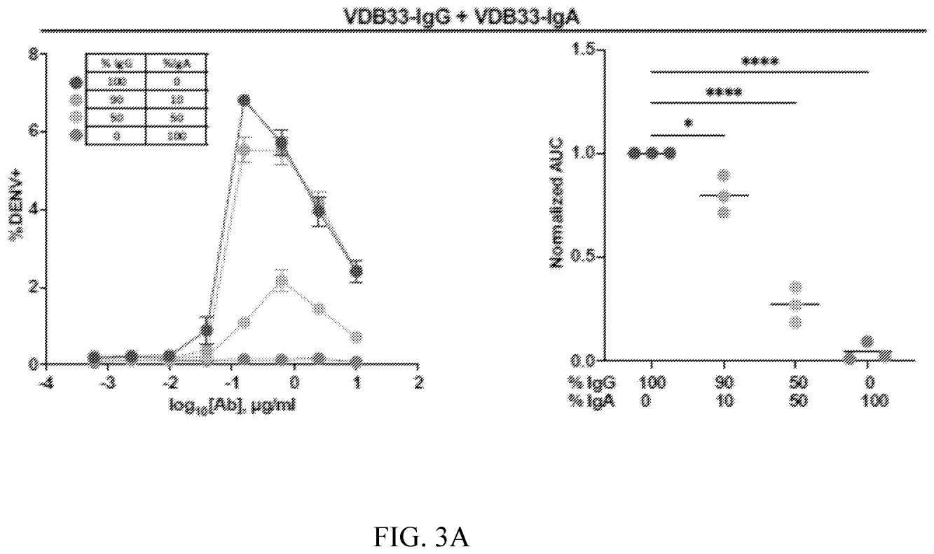

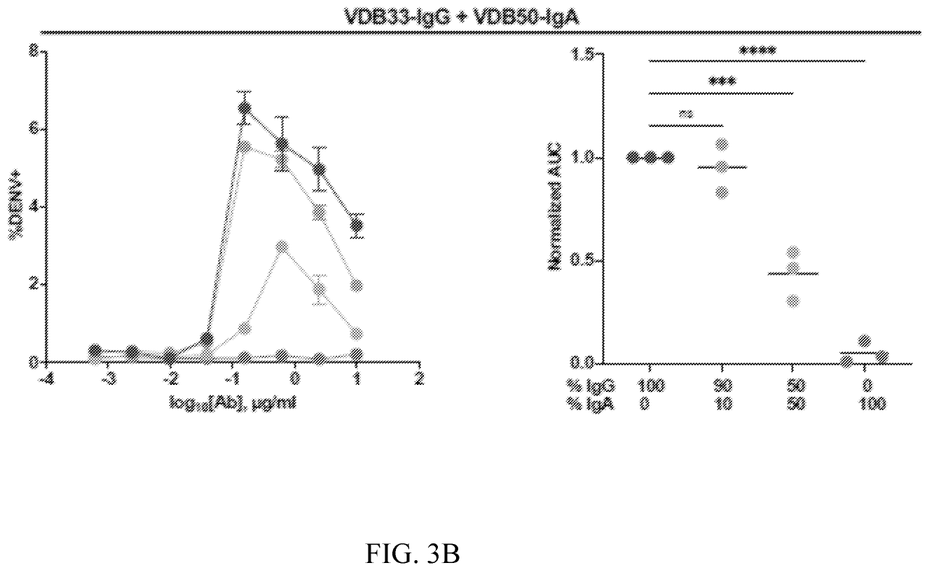

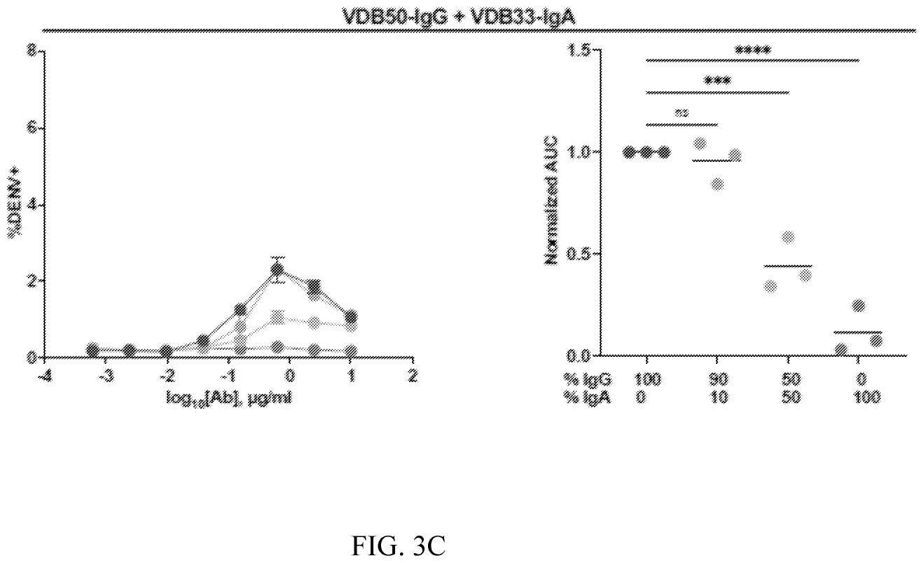

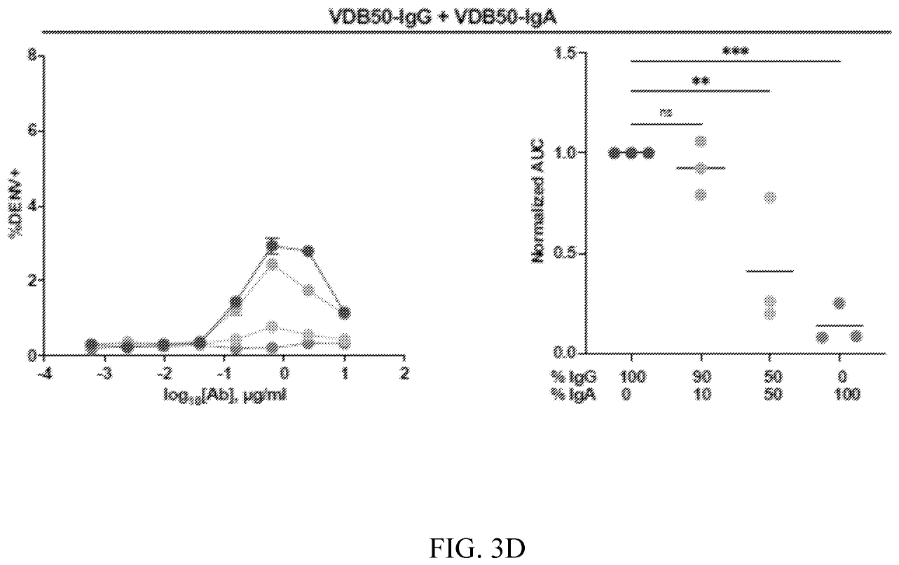

The same K562 ADE assay as previously described but used a fractional IgG1/IgA1 replacement strategy wherein the total amount of antibody remained the same across the different titration schemes but the ratio of IgG1 to IgA1 was varied from 100:0 to 0:100. The fractional addition of DENV-reactive IgA1 significantly reduced the ADE activity observed in cultures containing either VDB33-IgG1 or VDB50-IgG1 (See e.g., FIGS. 3A-3D). While both VDB33-IgA1 and VDB50-IgA1 were capable of antagonizing IgG1-mediated ADE of DENV −3, the highly avid yet non-enhancing VDB33-IgA1 antibody was capable of dramatically blunting IgG1-mediated ADE even when used at low fractional concentrations. Of note, the addition of DENV-reactive IgA1 to these ADE assays does not appear to shift the antibody dilution at which maximal ADE activity is observed for any of the cultures. Rather, the addition of DENV-reactive IgA1 reduces the magnitude of infection achieved at any given antibody dilution. These results are consistent with IgA1 actively antagonizing IgG1 mediated ADE by competing with DENV-reactive IgG1 for the same viral epitopes.

More specifically, it is noted that FIGS. 3A-3D depict homotypic and 30 heterotypic monoclonal IgA1 antagonizes IgG-mediated antibody-dependent enhancement. FIG. 3A depicts DENV −3 ADE activity of VDB33-IgG when antagonized with VDB33-IgA. Total antibody concentration for each dilution point was held constant, with varying ratios of VDB33-IgG and VDB33-IgA as indicated. AUC of each ADE titration was calculated and normalized to that of the 100% IgG condition. FIG. 3B depicts DENV −3 ADE activity of VDB33-IgG when antagonized with VDB50-IgA. The AUC of each ADE titration was calculated and normalized to that of the 100% VDB33-IgG condition. FIG. 3C depicts DENV −3 ADE activity of VDB50-IgG when antagonized with VDB33-IgA. AUC of each ADE titration was calculated and normalized to that of the 100% VDB50-IgG condition FIG. 3D depicts DENV −3 ADE activity of VDB50-IgG when antagonized with VDB50-IgA. AUC of each ADE titration was calculated and normalized to that of the 100% VDB33-IgG condition. Blue=100% IgG/0% IgA. Green=90% IgG1/10% IgA1. Orange=50% IgG1/50% IgA1. Red=0% IgG1/100% IgA1. * p<0.05, ** p<0.01, *** p<0.001, **** p<0.0001 1-way ANOVA with Dunnett correction for multiple comparisons

DENV-reactive IgA1 antagonizes DENV-immune serum mediated enhancement of DENV infection. A limitation of the analysis presented thus far is that all the monoclonal antibodies used in this analysis have the same antigen specificity; namely the fusion loop of the DENV E protein. Therefore, it is unclear what impact-if any-DENV-reactive IgA1 would have in the presence of a polyclonal IgG1 repertoire of divergent DENV antigen specificity. Therefore, we endeavored to determine how the presence of either VDB33-IgA1 or VDB50-IgA1 impacts the infection-enhancing potential of polyclonal/DENV-immune serum.

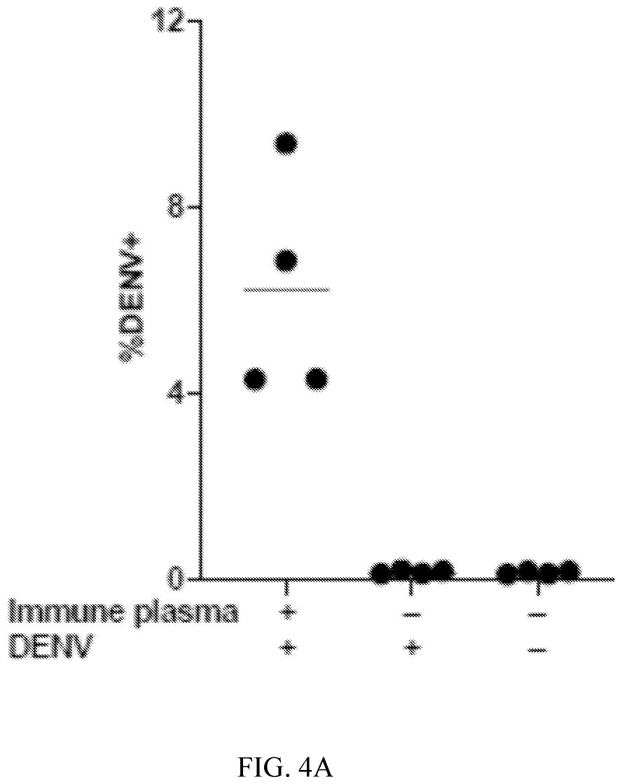

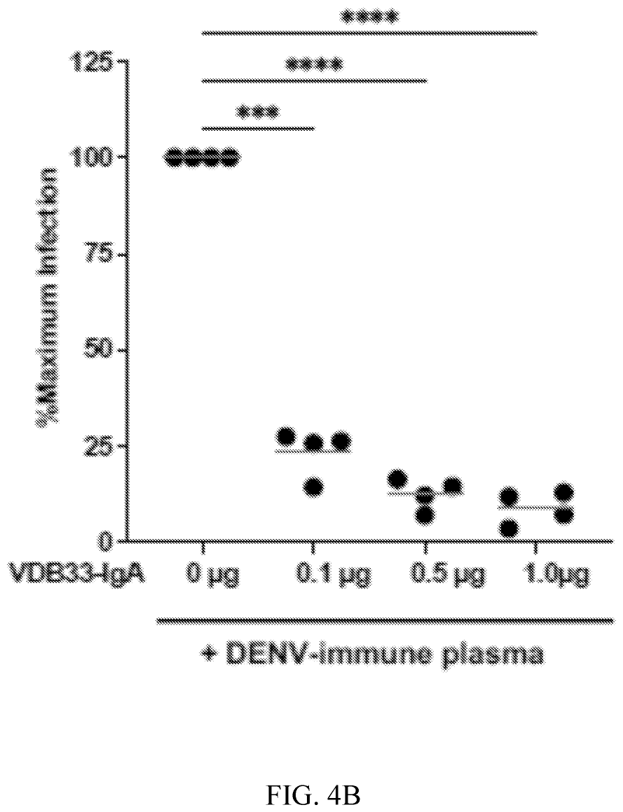

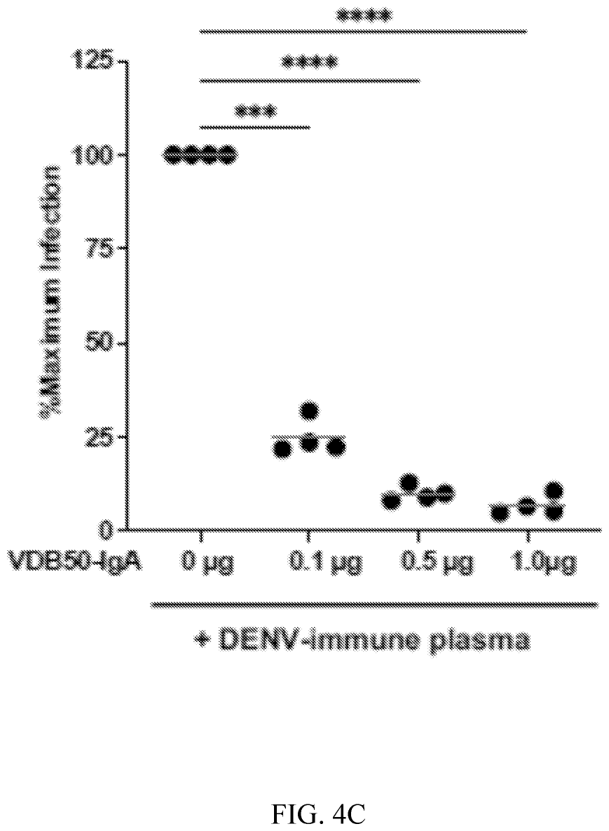

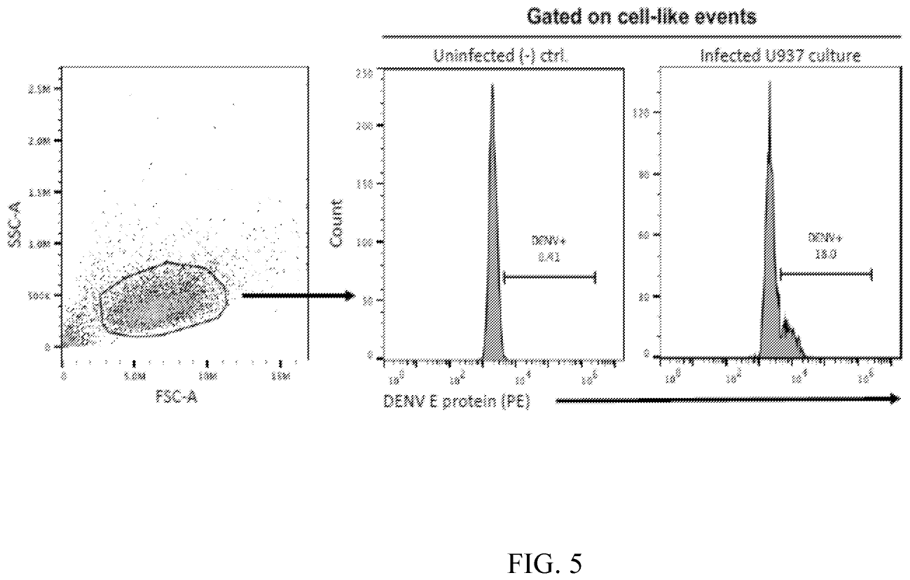

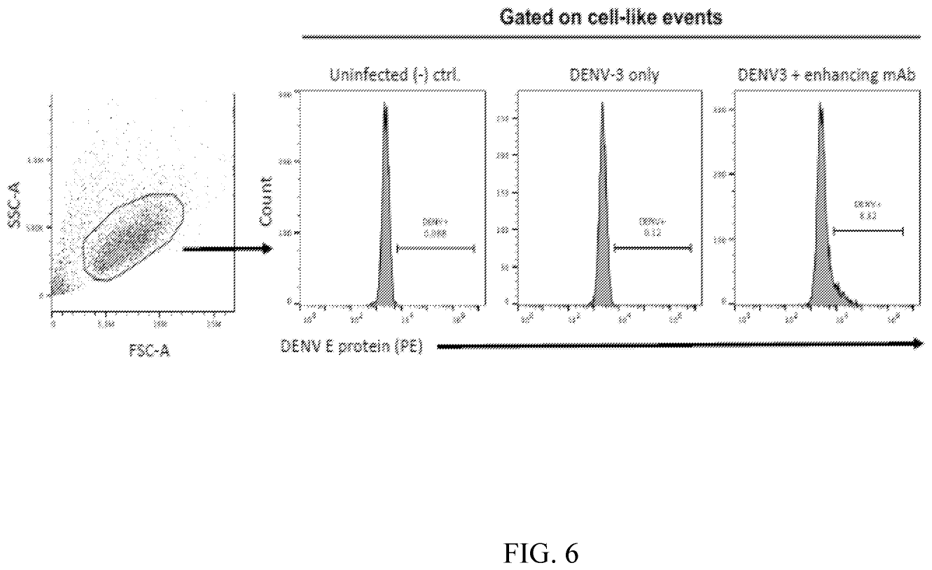

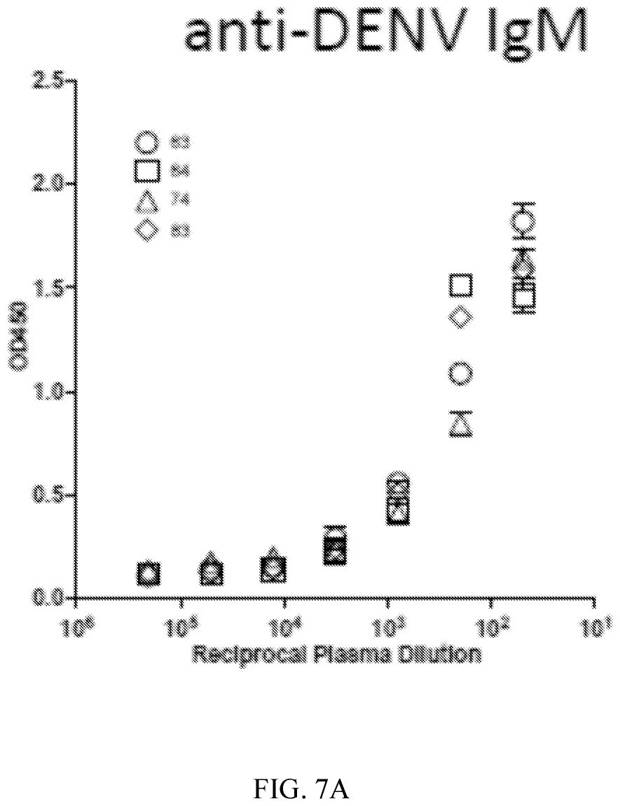

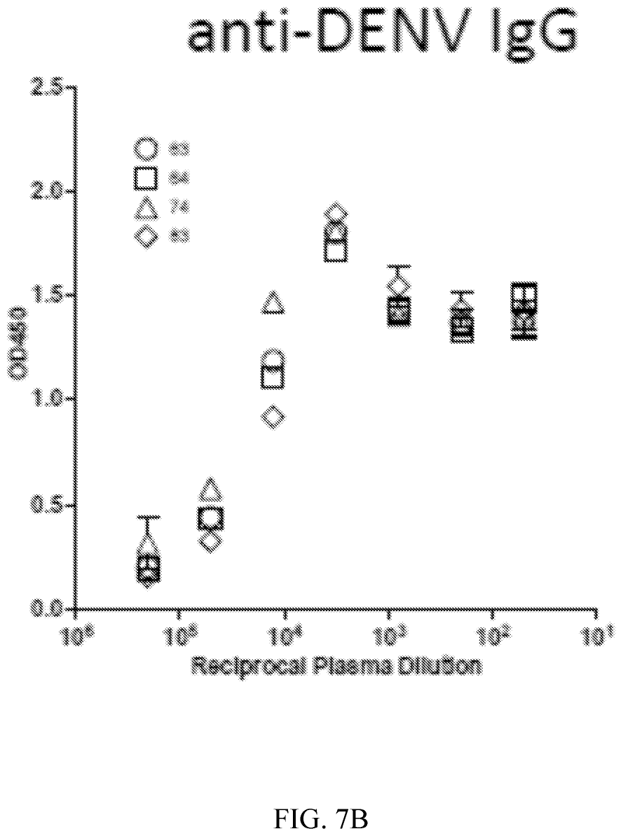

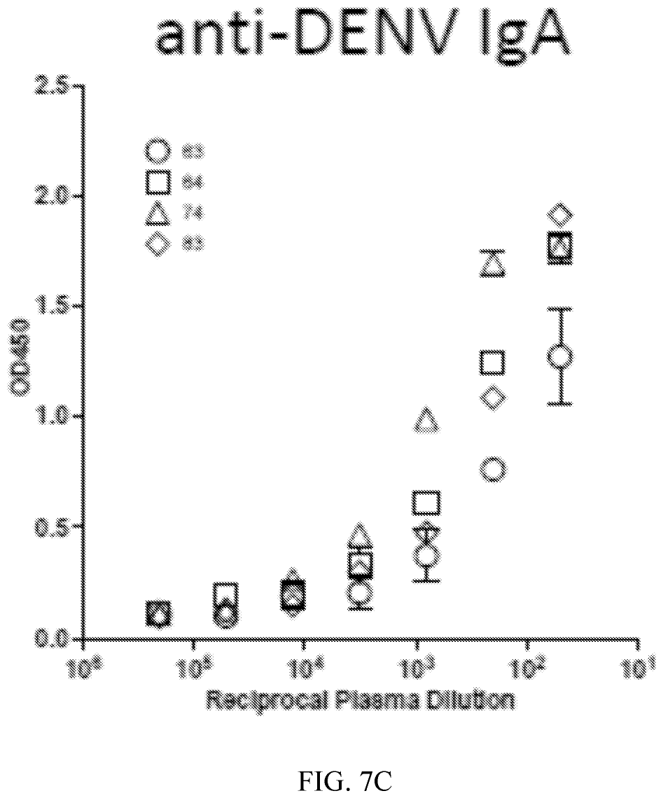

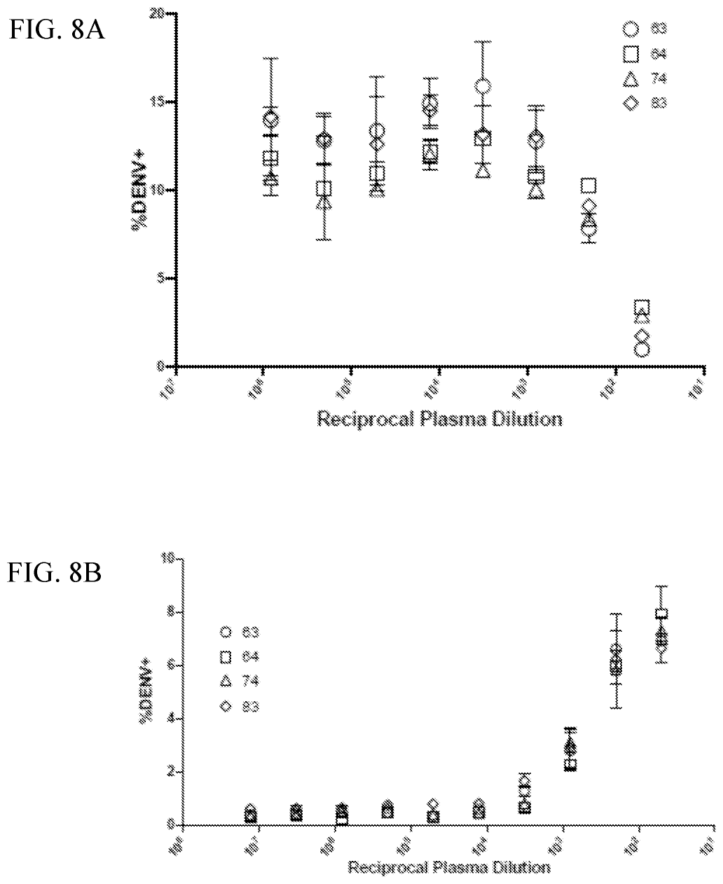

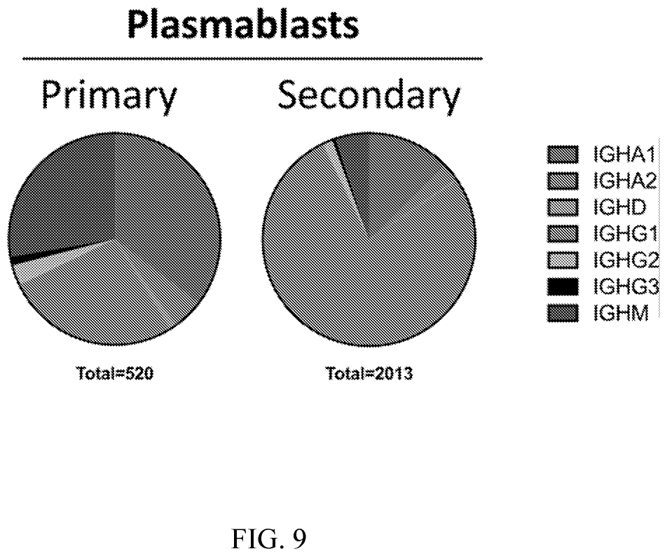

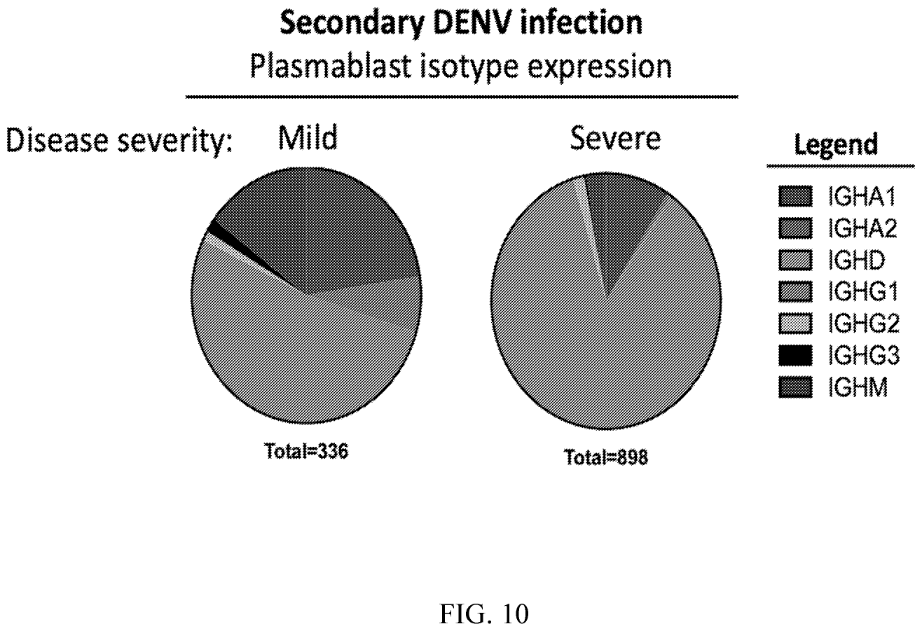

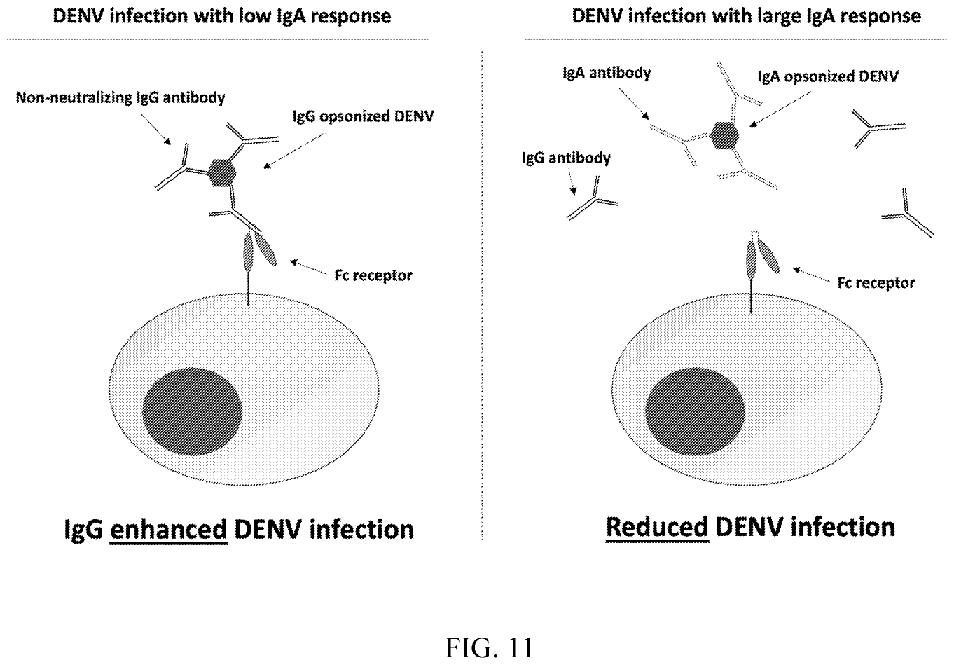

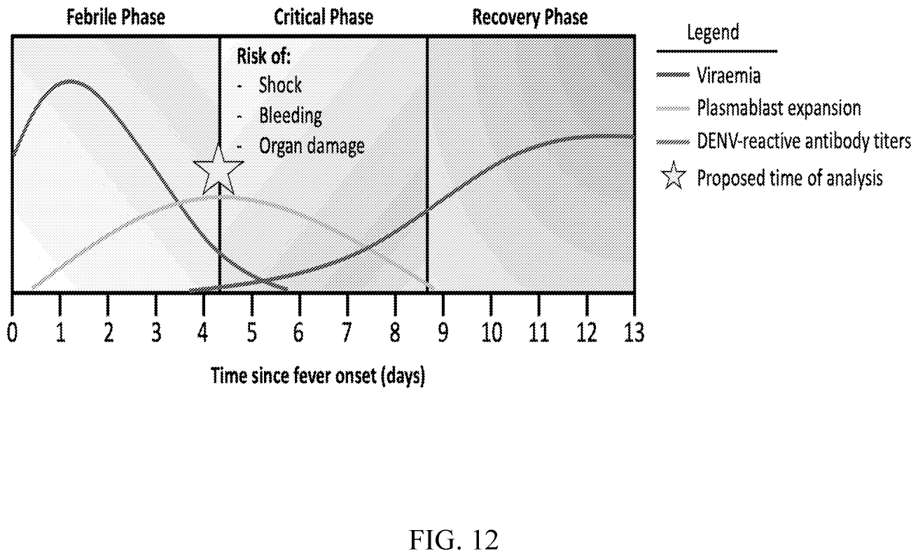

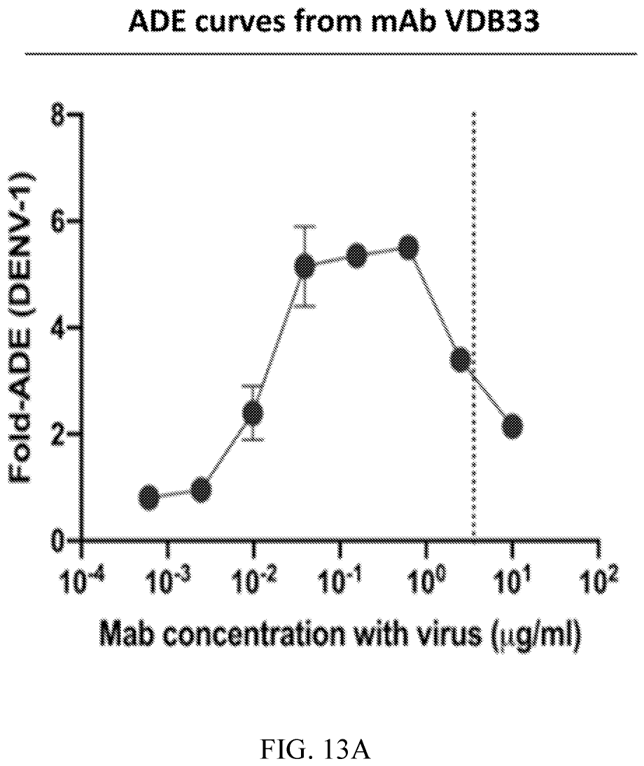

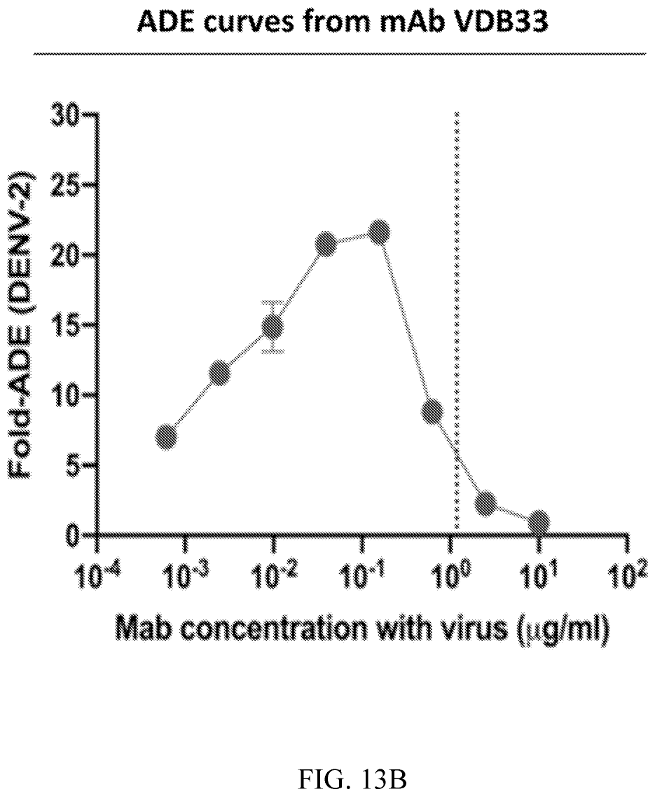

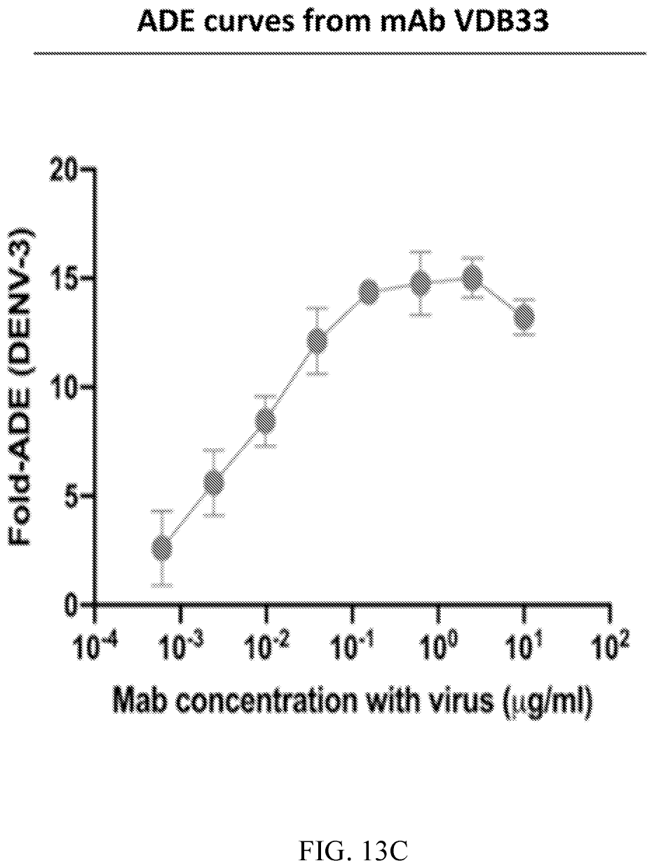

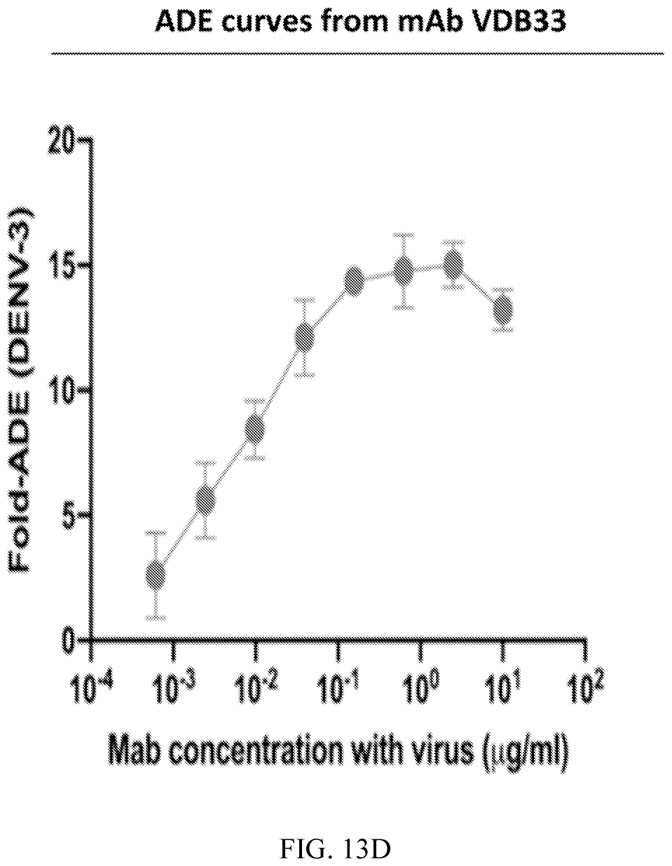

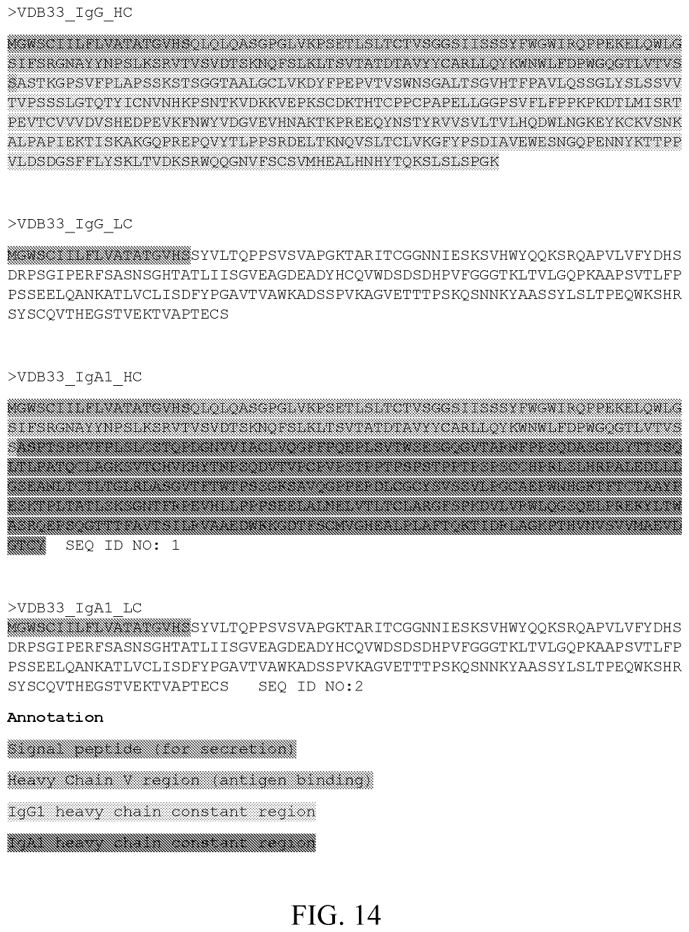

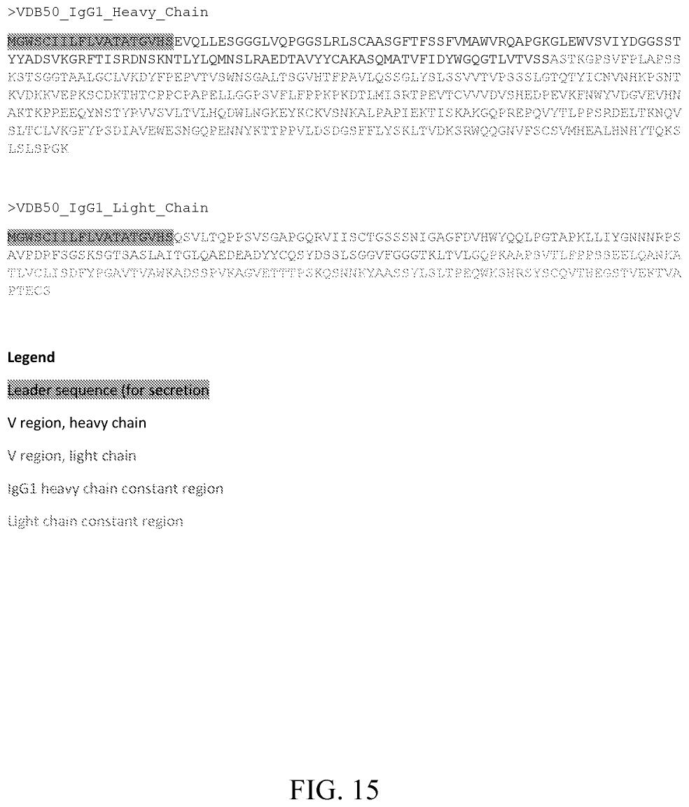

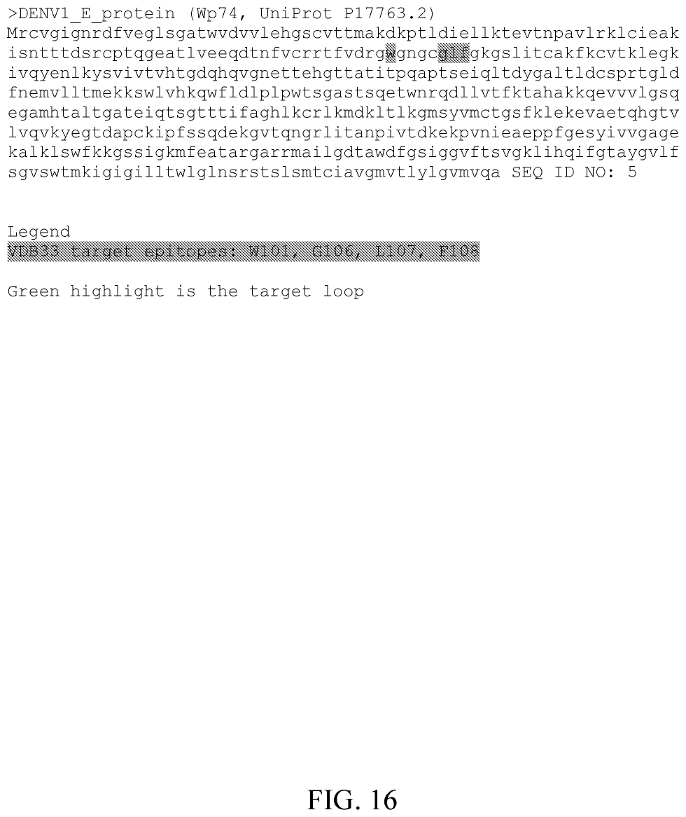

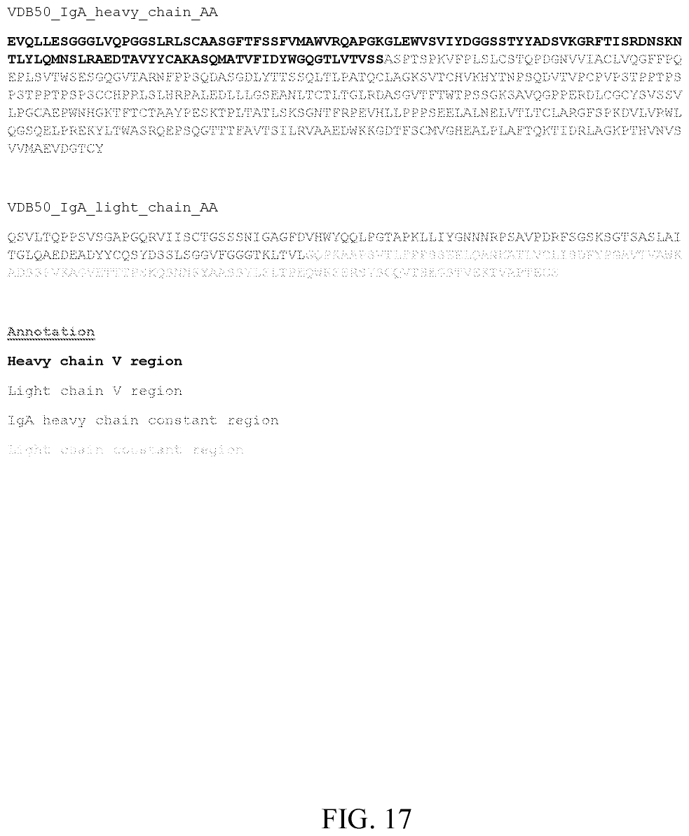

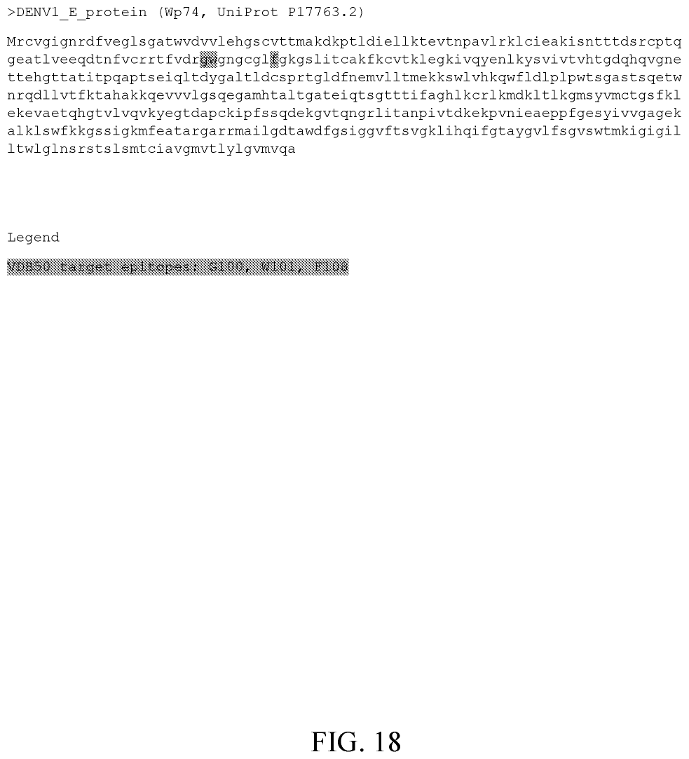

Plasma from DENV-immune donors were screened to identify samples with both high DENV −3 reactive IgG titers by ELISA as well as DENV −3 enhancing activity in the K562 ADE assay. Samples from four subjects were selected for additional analysis based on these criteria (See FIGS. 4A, 4B, and 4C, FIGS. 7A, 7B, and 7C, and FIGS. 8A and 8B).

VDB33-IgA1 or VDB50-IgA1 were then titrated into cultures containing this enhancing DENV-immune plasma to determine if IgA1 isotype monoclonal antibodies could antagonize polyclonal enhancement of DENV −3 infection.

Consistent with what was observed with IgG1 monoclonal antibodies, the addition of VDB33-IgA1 or VDB50-IgA1 significantly suppressed ADE-mediated K562 infection with DENV −3 (FIG. 4B, FIG. 4C). The additional of DENV-reactive IgA1 in these assays suppressed ADE-mediated infection by 75%-90% in a dose-dependent fashion, a result consistent with the concept that IgG antibodies targeting the fusion loop of the DENV E protein are particularly amenable to facilitating ADE activity [35]. These data also indicate that even modest concentrations of DENV-reactive IgA1 can significantly antagonize polyclonal IgG1-mediated enhancement of DENV infection, signifying that the presence of DENV E reactive IgA1 (especially fusion loop reactive IgA1) has the potential to significantly modulate DENV infection and associated immunopathogenesis.

FIGS. 4A-4C depicts Monoclonal IgA1 antagonizes ADE mediated by polyclonal DENV-immune plasma. FIG. 4A depicts DENV immune plasma enhances DENV −3 infection of K562 cells. Each datapoint represents a unique plasma donor (n=4). FIG. 4B depicts VDB33-IgA antagonizes in vitro enhancement of DENV-3 infection mediated by polyclonal DENV-immune serum. Serum used at a 1:50 dilution for ADE assay, n=4 unique plasma donors. The percentage of DENV-positive cells was normalized to that observed in the plasma-only condition. FIG. 4C depicts VDB50-IgA antagonizes in vitro enhancement of DENV −3 infection mediated by polyclonal DENV-immune serum. Serum used at a 1:50 dilution for ADE assay, n=4 unique plasma donors. The percentage of DENV-positive cells was normalized to that observed in the plasma-only condition. *** p<0.001, **** p<0.0001 1-way ANOVA with Dunnett correction for multiple comparisons

### DISCUSSION

In this study it was demonstrate that DENV-reactive IgA1 monoclonal antibodies can bind and neutralize DENV but are incapable of facilitating ADE. Furthermore, the addition of DENV-reactive IgA1 can significant blunt the DENV-infection enhancing activity of monoclonal and polyclonal DENV-reactive antibodies in a completive fashion. These results suggest an unappreciated role for DENV-reactive IgA during the humoral response to DENV infection and raise the potential that IgA could act as regulator of dengue severity and infection-attendant inflammation.

While the invention has been shown and described with reference to certain embodiments of the present invention thereof, it will be understood by those skilled in the art that various changes in form and details may be made therein without departing from the spirit and scope of the present disclosure.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed is:

1. An isolated monoclonal antibody, comprising: a heavy chain having an amino acid sequence of SEQ. ID NO. 1, wherein the heavy chain or a segment of the heavy chain comprises an Fc region characterized as IgA Fc domain.

2. The isolated monoclonal antibody of claim 1, wherein the IgA Fc domain is an IgA1 Fc domain.

3. The isolated monoclonal antibody of claim 1, wherein the IgA Fc domain is an IgA2 Fc domain.

4. The isolated monoclonal antibody of claim 1, wherein the antibody is chimeric or humanized.

5. The isolated monoclonal antibody of claim 1, further comprising: a light chain having an amino acid sequence of SEQ ID. NO. 2.

6. The isolated monoclonal antibody of claim 1, wherein the isolated monoclonal antibody binds an epitope of a Deng virus, wherein the epitope is characterized as a loop.

7. An isolated monoclonal antibody for targeting a fusion loop epitope of dengue virus, comprising:
a heavy chain having an amino acid sequence having at least 90% sequence identity to SEQ. ID NO. 1, wherein the heavy chain or a segment of the heavy chain comprises an Fc region characterized as IgA Fc domain; and
a light chain having an amino acid sequence having at least 90% sequence identity to SEQ ID. NO. 2.

8. The isolated monoclonal antibody of claim 7, wherein the heavy chain comprises an amino acid sequence having at least 95%, 97%, 98%, 99% sequence identity to SEQ. ID NO. 1.

9. The isolated monoclonal antibody of claim 7, wherein the light chain comprises an amino acid sequence having at least 95%, 97%, 98%, 99% sequence identity to SEQ. ID NO. 2.

10. An isolated monoclonal antibody, comprising: a heavy chain consisting of an amino acid sequence of SEQ. ID NO. 1; and
a light chain consisting of an amino acid sequence of SEQ ID NO: 2.

11. The isolated monoclonal antibody of claim 10, wherein the isolated monoclonal antibody binds a fusion loop epitope (FLE) of a Dengue virus.

12. An isolated monoclonal antibody, comprising: a heavy chain having an amino acid sequence having at least 90% sequence identity to SEQ. ID NO. 1, wherein the heavy chain or a segment of the heavy chain comprises an Fc region characterized as IgA Fc domain.

13. The isolated monoclonal antibody of claim 11, wherein the isolated monoclonal antibody binds an epitope of a Dengue virus, wherein the epitope is characterized as a loop.

14. A nucleic acid polymer encoding a monoclonal antibody, wherein the polymer comprises SEQ. ID NO. 1.

15. A nucleic acid polymer encoding a monoclonal antibody, wherein the polymer comprises SEQ. ID NO. 2.

16. A complementary deoxynucleotide (cDNA) sequence encoding an amino acid sequence having at least at least 90%, at least 95%, or at least 99% sequence identity to SEQ ID NO: 1.

17. A complementary deoxynucleotide (cDNA) sequence comprising a nucleic acid sequence of SEQ ID NO: 3.

18. A complementary deoxynucleotide (cDNA) sequence encoding an amino acid sequence having at least at least 90%, at least 95%, or at least 99% sequence identity to SEQ ID NO: 2.

19. A complementary deoxynucleotide (cDNA) sequence comprising a nucleic acid sequence of SEQ ID NO: 4.

20. A method for preventing or treating a dengue viral infection, the method comprising: administering a therapeutically effective amount of the monoclonal antibody of any of claims 1-13 to a subject in need thereof under conditions effective to treat the viral infection.

21. The method according to claim 20, wherein the method is used for preventing or treating antibody-dependent enhancement of a viral infection.

22. A method for preventing or treating antibody-dependent enhancement of a viral infection, the method comprising:
administering a therapeutically effective amount of a monoclonal antibody comprising a heavy chain or a segment of the heavy chain comprising an Fc region characterized as IgA Fc domain to a subject in need thereof under conditions effective to treat the viral infection, wherein the IgA Fc domain is characterized as an isotypic commutation.

23. The method according to claim 22, wherein the viral infection is any one of respiratory syncytial virus (RSV), influenza, and coronavirus infection, or a combination thereof.

24. The method according to claim 20, wherein the viral infection is a flavivirus infection.

25. The method according to claim 24, wherein the flavivirus infection is a Zika virus.

26. The method according to claim 25, wherein the flavivirus infection is a dengue virus.

27. A method for preventing or treating antibody-dependent enhancement of a viral infection, the method comprising:
administering a therapeutically effective amount of a monoclonal antibody comprising a heavy chain or a segment of the heavy chain comprising an Fc region characterized as IgA Fc domain to a subject in need thereof under conditions effective to treat the viral infection, wherein the IgA Fc domain is formed by class-switch recombination.

28. A method for preventing or treating antibody-dependent enhancement of a flavivirus infection, the method comprising:
administering a therapeutically effective amount of a monoclonal antibody of:
a) a monoclonal antibody encoded by a DNA polymer of claims 14 and 15;
b) a cDNA of claims 16 and 17; or
c) a monoclonal antibody of claims 1-13.

29. The method of claim 28, where in the monoclonal antibody is disposed with a serum comprising one or more additional antibodies or fragments thereof directed against dengue virus or bind one or more epitopes of dengue virus.

30. A method for preventing or treating antibody-dependent enhancement of a viral infection, the method comprising:
administering a therapeutically effective amount of an antibody comprising a heavy chain or a segment of the heavy chain comprising an Fc region characterized as IgA Fc domain to a subject in need thereof under conditions effective to prevent or treat antibody-dependent enhancement of a viral infection.

31. The method according to claim 30, wherein the viral infection is any one of respiratory syncytial virus (RSV), influenza, and coronavirus infection, or a combination thereof.

32. The method according to claim 30, wherein the viral infection is a flavivirus infection.

33. The method according to claim 32, wherein the flavivirus infection is a Zika virus.

34. The method according to claim 33, wherein the flavivirus infection is a dengue virus.

35. The method of claim 30, further comprising, increasing a concentration of antibodies characterized as IgA in a serum comprising one or more additional antibodies.

36. The method of claim 30, wherein the antibody is a wild-type IgA isoform that binds dengue protein E.

37. The method of claim 30, wherein the antibody is monoclonal.

38. The method of claim 30, wherein the antibody is synthetic, formed by recombinant technology, or characterized as isolated.

39. A method for preventing or treating antibody-dependent enhancement of a viral infection, the method comprising:
administering a therapeutically effective amount of a monoclonal antibody to a subject in need thereof under conditions effective to prevent or treat antibody-dependent enhancement of a viral infection, wherein the monoclonal antibody is characterized as an IgA isoform.

40. The method of claim 39, wherein the monoclonal antibody comprises a heavy chain or a segment of the heavy chain comprising an Fc region characterized as IgA Fc domain.

41. The method of claim 40, wherein the Fc region characterized as IgA Fc domain comprises an amino acid sequence of SEQ ID NO: 1.

42. The method of claim 40, wherein the Fc region characterized as IgA Fc domain comprises an amino acid segment between S31 and Y475 of SEQ ID NO. 1, an amino acid segment between G51 and Y475 of SEQ ID NO: 1, an amino acid segment between L101, or an amino acid segment between A181 and Y475, wherein SEQ ID NO: 1 is used for numbering.


SEQUENCE LISTING (11 sequences)

SEQ ID NO: 1 - protein, 475 aa, synthetic construct
  REGION 1..475: note = Synthetic sequence
QLQLQASGPGLVKPSETLSLTCTVSGGSIISSSYFWGWIRQPPEKELQWLGSIFSRGNAYYNPSLKSRVTVSVDTSKNQFSLKLTSVTATDTAVYYCARLLQYKWNWLFDPWGQGTLVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 214 aa, synthetic construct
  REGION 1..214: note = Synthetic sequence
SYVLTQPPSVSVAPGKTARITCGGNNIESKSVHWYQQKSRQAPVLVFYDHSDRPSGIPERFSASNSGHTATLIISGVEAGDEADYHCQVWDSDSDHPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - DNA, 1485 bp, synthetic construct
  misc_feature 1..1485: note = Synthetic sequence
  source 1..1485: mol_type = other DNA
atgggctggtcctgcatcattctgtttctggtggccacagccaccggcgtgcactctcaactgcaactgcaggcttctggccctggcctggtcaagccttctgagacactgagcctgacctgtaccgtgtctggcggcagcattatcagcagcagctacttctggggctggatcagacagcctcctgagaaagaactgcagtggctgggcagcatcttctccagaggcaacgcctactacaaccccagcctgaagtccagagtgaccgtgtccgtggacaccagcaagaaccagttctccctgaagctgaccagcgtgaccgccacagataccgccgtgtactactgtgccagactgctgcagtacaagtggaactggctgttcgacccttggggccagggaacactggtcacagtgtctagcgcctctccaacaagccccaaggtgttccctctgagcctgtgtagcacacagcccgacggcaatgtcgtgatcgcttgtctggtgcagggattcttcccacaagagcccctgtccgtgacttggagcgaatctggacagggcgtgacagccagaaacttcccacctagccaggatgccagcggcgatctgtacacaacaagcagccagctgaccctgcctgccacacaatgtctggccggcaagtctgtgacctgccacgtgaagcactacaccaatccaagccaggacgtgaccgtgccttgtcctgtgcctagcacacctcctacaccttctccaagcacaccaccaactccatctccatcctgctgtcaccccaggctgtctctgcatagacccgctctggaagatctgctgctgggctctgaggccaacctgacatgtacactgaccggcctgagagatgcctccggcgtgacctttacatggacacctagctctggcaagagcgccgttcagggacctcctgaaagggatctgtgcggctgttacagcgtgtcctctgtgctgcctggatgtgccgagccttggaatcacggcaagacctttacctgcaccgccgcctatcctgagagcaagacacctctgacagccacactgagcaagagcggcaacaccttcagacccgaagtgcatctgctgcctccaccatctgaagaactggccctgaacgagctggtcacactgacatgtctggctagaggcttcagccctaaggacgtgctcgtcagatggctgcagggctctcaagagctgcctagagagaagtacctgacctgggccagcagacaagagccttctcagggcaccaccacctttgccgtgaccagcattctgagagtggccgccgaggattggaagaagggcgataccttcagctgcatggtcggacacgaagccctgcctctggccttcacacagaaaaccatcgatcggctggccggaaagcccacacatgtgaatgtgtccgtcgtgatggccgaggtggacggcacatgttattga

SEQ ID NO: 4 - DNA, 702 bp, synthetic construct
  misc_feature 1..702: note = Synthetic sequence
  source 1..702: mol_type = other DNA
atgggatggtcatgtattattctgtttctggtcgcaactgcaaccggcgtgcatagcagctacgtgctgacacagcctccatccgtgtctgtggcccctggaaagaccgccagaatcacatgcggcggcaacaacatcgagagcaagagcgtgcactggtatcagcagaagtccagacaggcccctgtgctggtgttctacgaccacagcgatagacccagcggcatccccgagagattcagcgcctctaatagcggccacaccgccacactgatcatctctggtgttgaggccggcgacgaggccgattaccattgccaagtgtgggacagcgacagcgatcaccctgtttttggcggaggcaccaagctgacagtgctggggcagcccaaggccgctcctagcgtgacactgtttcccccttcctccgaggagctgcaggccaacaaggccaccctggtgtgcctgatctccgacttctatcctggcgccgtgacagtggcctggaaggctgattctagcccagtgaaggctggcgtggagaccacaaccccctccaagcagtctaacaataagtatgccgcttcctcttacctgagcctgacaccagagcagtggaagtcccaccggtcttacagctgccaggtcactcacgaaggctctaccgtggaaaagacagtcgcacccaccgaatgctcatga

SEQ ID NO: 5 - protein, 495 aa, synthetic construct
  REGION 1..495: note = synthetic sequence
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 6 - DNA, 1417 bp, synthetic construct
  misc_feature 1..1417: note = Synthetic sequence
  source 1..1417: mol_type = other DNA
atgggctggtcatgcattattctgtttctggtcgcaactgctacaggcgtgcatagtgaagtgcagctgctggaatctggcggaggactggttcaacctggcggctctctgagactgtcttgtgccgccagcggcttcaccttcagcagctttgtgatggcctgggtccgacaggcccctggcaaaggacttgaatgggtgtccgtgatctacgacggcggcagcagcacctactacgccgattctgtgaagggcagattcaccatcagccgggacaacagcaagaacaccctgtacctgcagatgaacagcctgagagccgaggacaccgccgtgtactattgtgccaaggccagccagatggccaccgtgttcatcgattattggggccagggcaccctggtcaccgtttcttctgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataagctt

SEQ ID NO: 7 - DNA, 711 bp, synthetic construct
  misc_feature 1..711: note = synthetic sequence
  source 1..711: mol_type = other DNA
atgggatggtcatgtattattctgtttctggtcgcaactgcaaccggcgtgcatagccagtctgtgctgacacagcctccatctgtgtctggcgctccaggccagagagtgatcatcagctgtacaggcagcagcagcaacatcggagccggctttgacgtgcactggtatcagcagctgcctggcacagcccctaaactgctgatctacggcaacaacaacagacccagcgccgtgcctgatagattcagcggctctaagagcggcacatctgccagcctggccattactggactgcaggccgaagatgaggccgactactactgccagagctacgacagctctctgtctggcggagtttttggcggaggcaccaagctgacagtgctggggcagcccaaggccgctcctagcgtgacactgtttcccccttcctccgaggagctgcaggccaacaaggccaccctggtgtgcctgatctccgacttctatcctggcgccgtgacagtggcctggaaggctgattctagcccagtgaaggctggcgtggagaccacaaccccctccaagcagtctaacaataagtatgccgcttcctcttacctgagcctgacaccagagcagtggaagtcccaccggtcttacagctgccaggtcactcacgaaggctctaccgtggaaaagacagtcgcacccaccgaatgctcatga

SEQ ID NO: 8 - DNA, 1422 bp, synthetic construct
  misc_feature 1..1422: note = Synthetic sequence
  source 1..1422: mol_type = other DNA
gaagttcagctgcttgagtctggcggcggactggttcaacctggcggatctctgagactgagctgtgccgccagcggcttcaccttcagcagctttgtgatggcctgggtccgacaggcccctggcaaaggacttgaatgggtgtccgtgatctacgacggcggcagcagcacctactacgccgattctgtgaagggcagattcaccatcagccgggacaacagcaagaacaccctgtacctgcagatgaacagcctgagagccgaggacaccgccgtgtactattgtgccaaggccagccagatggccaccgtgttcatcgattattggggccagggcaccctggtcaccgtgtcatctgctagccctacaagccccaaggtgttccctctgagcctgtgtagcacacagcccgacggcaatgtcgtgatcgcttgtctggtgcagggattcttcccacaagagcccctgtccgtgacttggagcgaatctggacagggcgtgaccgccagaaacttcccaccttctcaggatgccagcggcgacctgtacacaacaagcagccaactgaccctgcctgccacacagtgtctggccggaaagtctgtgacctgccacgtgaagcactacacaaaccccagccaggacgtgaccgtgccttgtcctgttcctagcacacctcctacaccttctccaagcacaccaccaactccatctccatcctgctgtcaccccagactgagcctgcatagacccgctctggaagatctgctgctgggctctgaggccaacctgacatgtacactgaccggcctgagagatgcctccggcgtgacctttacatggacacctagcagcggcaagagcgccgttcaaggacctcctgagagggatctgtgcggctgttacagcgtgtcctctgtgctgcctggatgtgccgagccttggaatcacggcaagaccttcacctgtaccgccgcctatcctgagagcaagacccctctgacagccacactgagcaagagcggcaacacctttcggcccgaagtgcatcttctgcctccacctagcgaagaactggccctgaatgagctggtcaccctgacatgcctggccagaggcttcagccctaaggatgtgctcgtcagatggctgcagggcagccaagagctgcccagagagaagtatctgacctgggccagcagacaagagcctagccagggaaccaccacctttgccgtgaccagcattctgagagtggccgccgaggattggaagaagggcgataccttcagctgcatggtcggacacgaagccctgccactggccttcacacagaaaaccatcgacagactggccggcaagcccacacatgtgaatgtgtctgtggtcatggccgaggtggacggcacatgttattga

SEQ ID NO: 9 - DNA, 654 bp, synthetic construct
  misc_feature 1..654: note = Synthetic sequence
  source 1..654: mol_type = other DNA
cagtctgtgctgacacagcctccatctgtgtctggcgctccaggccagagagtgatcatcagctgtacaggcagcagcagcaacatcggagccggctttgacgtgcactggtatcagcagctgcctggcacagcccctaaactgctgatctacggcaacaacaacagacccagcgccgtgcctgatagattcagcggctctaagagcggcacatctgccagcctggccattactggactgcaggccgaagatgaggccgactactactgccagagctacgacagctctctgtctggcggagtttttggcggaggcaccaagctgacagtgctggggcagcccaaggccgctcctagcgtgacactgtttcccccttcctccgaggagctgcaggccaacaaggccaccctggtgtgcctgatctccgacttctatcctggcgccgtgacagtggcctggaaggctgattctagcccagtgaaggctggcgtggagaccacaaccccctccaagcagtctaacaataagtatgccgcttcctcttacctgagcctgacaccagagcagtggaagtcccaccggtcttacagctgccaggtcactcacgaaggctctaccgtggaaaagacagtcgcacccaccgaatgctcatga

SEQ ID NO: 10 - protein, 473 aa, synthetic construct
  REGION 1..473: note = Synthetic sequence
EVQLLESGGGLVQPGGSLRLSCAASGFTFSSFVMAWVRQAPGKGLEWVSVIYDGGSSTYYADSVKGRFTISRDNSKNTLYLQMNSLRAEDTAVYYCAKASQMATVFIDYWGQGTLVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 11 - protein, 217 aa, synthetic construct
  REGION 1..217: note = Synthetic sequence
QSVLTQPPSVSGAPGQRVIISCTGSSSNIGAGFDVHWYQQLPGTAPKLLIYGNNNRPSAVPDRFSGSKSGTSASLAITGLQAEDEADYYCQSYDSSLSGGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS